(19) United States
(12) Patent Application Publication
(10) Pub. No.: US 2025/0354201 A1
(43) Pub. Date: Nov. 20, 2025

(54) FERROCENE LABELLED PRIMERS FOR ELECTROCHEMICAL DETECTION

(71) Applicant: Roche Molecular Systems, Inc., Pleasanton, CA (US)

(72) Inventor: Saurabh Sahar, Orange, CA (US)

(21) Appl. No.: 18/871,318

(22) PCT Filed: Jun. 7, 2023
(86) PCT No.: PCT/EP2023/065313

§ 371 Date: Dec. 3, 2024

Related U.S. Application Data
(60) Provisional application No. 63/349,980, filed on Jun. 7, 2022.

(51) Int. Cl. C12Q 1/6825; C12Q 1/6853; C12Q 1/70; G01N 27/327

(52) U.S. Cl. CPC C12Q 1/6825; C12Q 1/6853; C12Q 1/701; G01N 27/3276

(57) ABSTRACT

The present invention provides novel primers in which a ferrocene label is attached to the primer. The ferrocene label is incorporated into the amplification product. When the amplification product incorporating the ferrocene label is denatured, it can bind to a capture probe and the presence or absence of the ferrocene label can be detected via electrochemical detection. The system avoids the use of a signal probe in a sandwich assay as historically used during electrochemical detection.

### FIELD OF THE INVENTION

The present invention relates generally to the fields of electrochemical detection technology and molecular diagnostics.

### BACKGROUND OF THE INVENTION

Access to diagnostic tests is critical to help limit the spread of disease. Yet, during the COVID-19 pandemic, access to effective diagnostic tests was a significant problem especially during the initial outbreak in 2020. Accelerated development and scaled up manufacturing and delivery of COVID-19 tests was critical in the fight against COVID-19. There remains a need to quickly develop accurate, fast assays and manufacture those assays in order to get them quickly to market when additional COVID-19 variants and new infectious diseases arise.

### SUMMARY OF THE INVENTION

Provided herein are methods, devices, and systems for amplifying and detecting nucleic acids, such as DNA and/or RNA, using a signal primer for use in electrochemical detection systems.

Amplifying and detecting nucleic acids (such as DNA and/or RNA) using a signal primer accelerates the development of detection assays, improves their accuracy, speeds up the time to result and simplifies manufacturing in order to ensure that diagnostic kits can be quickly developed and delivered to patients in need. Rapid development and distribution of diagnostic kits will help reduce the spread of disease.

In a first aspect a method of detecting the presence or absence of a target nucleic acid in a sample is provided, the method comprising (a) combining a solution comprising target nucleic acid or suspected to comprise target nucleic acid with amplification reagents to amplify the target nucleic acid if present, the amplification reagents comprising a labeled primer capable of hybridizing to the target nucleic acid; (b) amplify the target nucleic acid to produce a double stranded labeled amplicon if the target nucleic acid is present; (c) incubating the double stranded labeled amplicon with exonuclease to form a single stranded labeled amplicon; (d) hybridizing the single stranded labeled amplicon with a first capture probe; and (e) detecting the presence or absence of the target nucleic acid in the sample using electrochemical detection. In some embodiments, the labeled primer, double stranded labeled amplicon, and/or single stranded labeled amplicon comprise at least one label selected from the group consisting of ferrocene, methylene blue or osmium. In certain embodiments, the at least one label is ferrocene. In some embodiments, the labeled primer comprises a ferrocene label on the 5′-end, on any one nucleotide of nucleotides 1 to 3 at the 5′-end, on the 3′-end, on any one nucleotide of nucleotides 1 to 3 at the 3′-end or on both, the 5′-and the 3′-end. In some embodiments, the labeled primer comprises a ferrocene label internal to the labeled primer. In some embodiments, the labeled primer, double stranded labeled amplicon, and/or single stranded labeled amplicon comprise a plurality of ferrocene labels at at least two locations. In some embodiments, the single stranded labeled amplicon comprises a first section capable of hybridizing to the first capture probe, a second section that is not capable of binding to the capture probe and a third section comprising the at least one label. In certain embodiments, the label is selected from the group consisting of ferrocene, methylene blue or osmium. In some embodiments, the labeled primer comprises a first section capable of hybridizing to the first capture probe, a second section comprising a linker and a third section comprising the at least one label, wherein the linker connects the first section and the third section. In some embodiments, the method further comprises separating the labeled primer and the single stranded labeled amplicon prior to electrochemical detection. In another embodiment, the method further comprises separating the labeled primer and the double stranded labeled amplicon prior to electrochemical detection. In some embodiments, the labeled primer consists of a first section capable of hybridizing to the capture probe, and a second section comprising the at least first label. In some embodiments, the amplification reagents comprise nucleic acid polymerase, deoxynucleotide triphosphates (dNTPs), reaction buffer necessary for the function of the nucleic acid polymerase and a divalent cation (such as, e.g., Mg²⁺ and/or Mn²⁺). In some embodiments, the target nucleic acid is a DNA. In some embodiments, the nucleic acid is an RNA and the method further comprises the step of reverse transcribing RNA to generate cDNA using a reverse transcriptase. In some embodiments, the nucleic acid polymerase is a DNA polymerase. In some embodiments, the nucleic acid polymerase further exhibits reverse transcriptase activity.

In a second aspect, a method of detecting the presence or absence of a target nucleic acid in a sample, the method comprising (a) receiving a sample; (b) extracting nucleic acids from the sample, wherein the nucleic acids are suspected of comprising target nucleic acid; (c) combining the nucleic acid with amplification reagents to amplify the target nucleic acid if present, the amplification reagents comprising a signal primer capable of hybridizing to the target nucleic acid; (d) amplify the target nucleic acid to produce a double stranded signal amplicon if the target nucleic acid is present; (e) incubating the double stranded signal amplicon with exonuclease to form a single stranded signal amplicon; (f) hybridizing the single stranded signal amplicon with a first capture probe; and (g) detecting the presence or absence of the target nucleic acid in the sample using electrochemical detection. In some embodiments, the signal primer comprises an electrochemically detectable label. In some embodiments, the signal primer comprises at least one label selected from the group consisting of ferrocene, methylene blue or osmium. In certain embodiments, the at least one label is ferrocene. Herein, the detectable label may act as a signaling moiety. In some embodiments, the signal primer does not bind/hybridize to the first capture probe. In some embodiments, a first portion of the single stranded signal amplicon binds/hybridizes to the first capture probe and a second portion of the single stranded signal amplicon binds/hybridizes to a second capture probe. In some embodiments, the labeled primer, double stranded labeled amplicon, and/or single stranded labeled amplicon comprise at least one label selected from the group consisting of ferrocene, methylene blue or osmium. In certain embodiments, the at least one label is ferrocene. In some embodiments, the labeled primer comprises a ferrocene label on the 5′-end, on any one nucleotide of nucleotides 1 to 3 at the 5′-end, on the 3′-end, on any one nucleotide of nucleotides 1 to 3 at the 3′-end or on both, the 5′-and the 3′-end. In some embodiments, the labeled primer comprises a ferrocene label internal to the labeled primer. In some embodiments, the labeled primer, double stranded labeled amplicon, and/or single stranded labeled amplicon comprise a plurality of ferrocene labels at at least two locations. In some embodiments, the single stranded labeled amplicon comprises a first section capable of hybridizing to the first capture probe, a second section that is not capable of binding to the capture probe and a third section comprising the at least one label. In certain embodiments, the label is selected from the group consisting of ferrocene, methylene blue or osmium. In some embodiments, the labeled primer comprises a first section capable of hybridizing to the first capture probe, a second section comprising a linker and a third section comprising the at least one label, wherein the linker connects the first section and the third section. In some embodiments, the method further comprises separating the labeled primer and the single stranded labeled amplicon prior to electrochemical detection. In another embodiment, the method further comprises separating the labeled primer and the double stranded labeled amplicon prior to electrochemical detection. In some embodiments, the labeled primer consists of a first section capable of hybridizing to the capture probe, and a second section comprising the at least first label. In some embodiments, the amplification reagents comprise nucleic acid polymerase, deoxynucleotide triphosphates (dNTPs), reaction buffer necessary for the function of the nucleic acid polymerase and a divalent cation (such as, e.g., Mg²⁺ and/or Mn²⁺). In some embodiments, the target nucleic acid is a DNA. In some embodiments, the nucleic acid is an RNA and the method further comprises the step of reverse transcribing RNA to generate cDNA using a reverse transcriptase. In some embodiments, the nucleic acid polymerase is a DNA polymerase. In some embodiments, the nucleic acid polymerase further exhibits reverse transcriptase activity.

In a third aspect, a process for detecting the presence of a single-stranded or double-stranded nucleic acid of interest in a sample, said process comprising the steps of (a) providing (i) a sample suspected of containing said nucleic acid of interest, (ii) a nucleic acid primer that comprises a nucleic acid sequence complementary to at least a portion of said nucleic acid of interest, and a first electrochemically detectable label and (iii) reagents for carrying out nucleic acid strand extension; (b) forming a reaction mixture comprising (i), (ii), and (iii) above; (c) contacting under hybridization conditions the nucleic acid primer with the nucleic acid of interest if present; (d) extending the nucleic acid primer, thereby incorporating the electrochemically detectable label into an amplicon to form an electrochemically labeled amplicon if said nucleic acid of interest is present; (e) denaturing the electrochemically labeled amplicon; (f) hybridizing the electro-chemically labeled amplicon with a capture probe bound to an electrode surface; (g) detecting the presence of the nucleic acid of interest by detecting energy transfer between the electrochemically labeled amplicon and the electrode surface. In some embodiments, the electrochemically detectable label is selected from the group consisting of ferrocene, methylene blue or osmium. In certain embodiments, the at least one label is ferrocene. Herein, the detectable label may act as a signaling moiety. In some embodiments, the nucleic acid primer comprises a first portion that binds/hybridizes to the capture probe and a second portion that does not bind/hybridize to the capture probe. In some embodiments, the nucleic acid primer further comprises a second electrochemically detectable label and the first electrochemically detectable label and the second electrochemically detectable label are different. In some embodiments, the nucleic acid primer is a signal primer that does not bind/hybridize to the first capture probe. In some embodiments, a first portion of the single stranded signal amplicon binds/hybridizes to the first capture probe and a second portion of the single stranded signal amplicon binds/hybridizes to a second capture probe. In some embodiments, the nucleic acid primer, double stranded labeled amplicon, and/or single stranded labeled amplicon comprise at least one label selected from the group consisting of ferrocene, methylene blue or osmium. In certain embodiments, the at least one label is ferrocene. In some embodiments, the nucleic acid primer comprises a ferrocene label on the 5′-end, on any one nucleotide of nucleotides 1 to 3 at the 5′-end, on the 3′-end, on any one nucleotide of nucleotides 1 to 3 at the 3′-end or on both, the 5′-and the 3′-end. In some embodiments, the nucleic acid primer comprises a ferrocene label internal to the labeled primer. In some embodiments, the nucleic acid primer, double stranded labeled amplicon, and/or single stranded labeled amplicon comprise a plurality of ferrocene labels at at least two locations. In some embodiments, the single stranded labeled amplicon comprises a first section capable of hybridizing to the first capture probe, a second section that is not capable of binding to the capture probe and a third section comprising the at least one label. In certain embodiments, the label is selected from the group consisting of ferrocene, methylene blue or osmium. In some embodiments, the nucleic acid primer comprises a first section capable of hybridizing to the first capture probe, a second section comprising a linker and a third section comprising the at least one label, wherein the linker connects the first section and the third section. In some embodiments, the process further comprises separating the nucleic acid primer and the single stranded labeled amplicon prior to electrochemical detection. In another embodiment, the process further comprises separating the nucleic acid primer and the double stranded labeled amplicon prior to electrochemical detection. In some embodiments, the nucleic acid primer consists of a first section capable of hybridizing to the capture probe, and a second section comprising the at least first label. In some embodiments, the reagents for carrying out nucleic acid strand extension comprise nucleic acid polymerase, deoxynucleotide triphosphates (dNTPs), reaction buffer necessary for the function of the nucleic acid polymerase and a divalent cation (such as, e.g., Mg²⁺ and/or Mn²⁺). In some embodiments, the target nucleic acid is a DNA. In some embodiments, the nucleic acid is an RNA and the method further comprises the step of reverse transcribing RNA to generate cDNA using a reverse transcriptase. In some embodiments, the nucleic acid polymerase is a DNA polymerase. In some embodiments, the nucleic acid polymerase further exhibits reverse transcriptase activity.

### BRIEF DESCRIPTION OF THE DRAWINGS

FIG. 1a shows a typical sandwich assay used in electrochemical detection. In FIG. 1a, the sandwich comprises three main elements, the capture probe (2), the signal probe (3) and the target (4). The target can be synonymous with a PCR amplicon sequence in nucleic acid embodiments. The target can have a portion (4b) which binds or hybridizes specifically to a desired portion of the signal probe (3b), a portion (4a) which binds or hybridizes to a corresponding capture probe portion (2b) and optionally one or more flanking portions, e.g., (4c). The capture probe can include a linker (2a) that links, joins or binds the capture probe (2) to the electrode surface (1). As pictured, the signal probe has a detectable-labeled portion or labels (3a) that are in close proximity to the electrode surface. The labeled portion 3a is conjugated or internal to the signal probe-binding portion (3b). Not pictured is a self-assembled monolayer (SAM), which is also attached to the electrode surface in some variants via one or more linkers in similar format to the capture probe linkers (2a), and which serves to prevent or lessen undesired electron transfer events (“noise”) to the electrode surface.

FIG. 1b shows a typical signal complex as disclosed herein. In FIG. 1b, the assay comprises just two main elements, the capture probe (2), and the target (4). The target can be synonymous with a PCR amplicon sequence in nucleic acid embodiments. The target can have a portion (4a), which binds or hybridizes specifically to a desired portion of the capture probe portion (2b). Not shown, the target (4) can optionally comprise one or more portions flanking the capture probe. The capture probe can include a linker (2a) that links, joins or binds the capture probe (2) to the electrode surface (1). As pictured, the target has a detectable-labeled portion or labels (3a) that are in close proximity to the electrode surface. The labeled portion 3a is conjugated to the target at the signal-binding portion (4b). Not pictured is a self-assembled monolayer (SAM), which is also attached to the electrode surface in some embodiments via one or more linkers in similar format to the capture probe linkers (2a), and which serves to prevent or lessen undesired electron transfer events (“noise”) to the electrode surface.

FIG. 2 shows embodiments of the signal primers disclosed herein. FIGS. 2a-2c show different embodiments of the signal primers disclosed herein. In some embodiments, a first signal moiety (which is shown as 3 circles) is on the 5′-end of the signal primer and a second signal moiety is conjugated internally to the signal primer itself (FIG. 2a). In some embodiments, the signal moiety (which is shown as 3 circles) is on the 5′-end of the signal primer (FIG. 2c). In some embodiments, the signal moiety (which is shown as 3 circles) is conjugated internally to the signal primer itself (FIG. 2b). Stated another way, a redox moiety is attached to the primer, which label can be introduced at the 5′-terminus or anywhere in the middle of the sequence. In FIGS. 2 (a-c), ‘ii’ is the detection moiety, e.g., ferrocene, and ‘i’ is the annealing region, e.g., the region that binds to the DNA template during PCR.

FIGS. 2d-2f show different embodiments of the single stranded signal amplicon disclosed herein. In some embodiments a first signal moiety (which is shown as 3 circles) is on the 5′-end of the single stranded signal amplicon and a second signal moiety is conjugated internally to the signal primer itself (FIG. 2d). In some embodiments, the signal moiety (which is shown as 3 circles) is on the 5′-end of the single stranded signal amplicon (FIG. 2f). In some embodiments, the signal moiety (which is shown as 3 circles) is conjugated internally to the single stranded signal amplicon (FIG. 2e). Stated another way, a redox moiety is attached to the single stranded signal amplicon, which label can be introduced at the 5′-terminus or anywhere in the middle of the sequence. In FIGS. 2 (d-f), ‘ii’ is the detection moiety, e.g., ferrocene, and ‘i’ is the annealing region, e.g., the region that binds to the capture probe during detection.

FIGS. 2g-2k show different embodiments of the double stranded signal amplicon disclosed herein. In some embodiments, a first signal moiety (which is shown as 3 circles) is on the 5′-end of the double stranded signal amplicon and a second signal moiety is conjugated internally to the signal primer itself. (FIG. 2g). In some embodiments, the signal moiety (which is shown as 3 circles) is on the 5′-end of the single stranded signal amplicon (FIG. 2i). In some embodiments, the signal moiety (which is shown as 3 circles) is conjugated or internal to the single stranded signal amplicon (FIG. 2h).

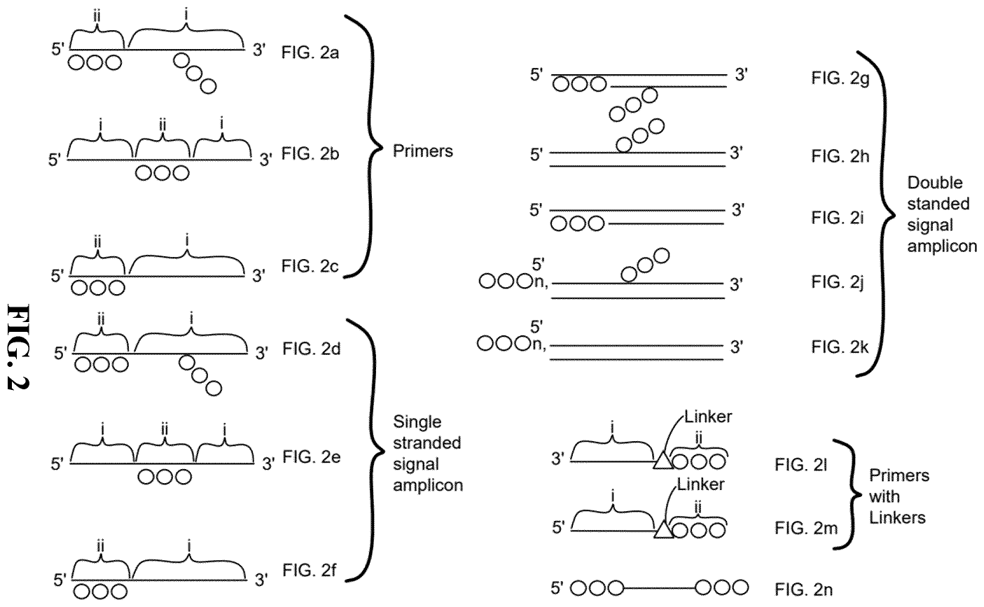

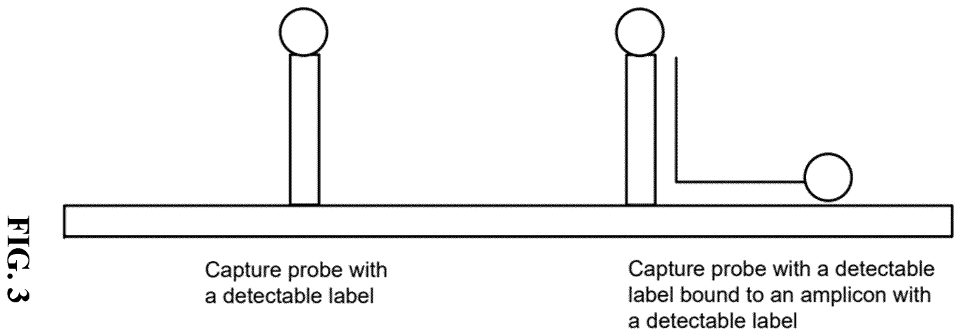

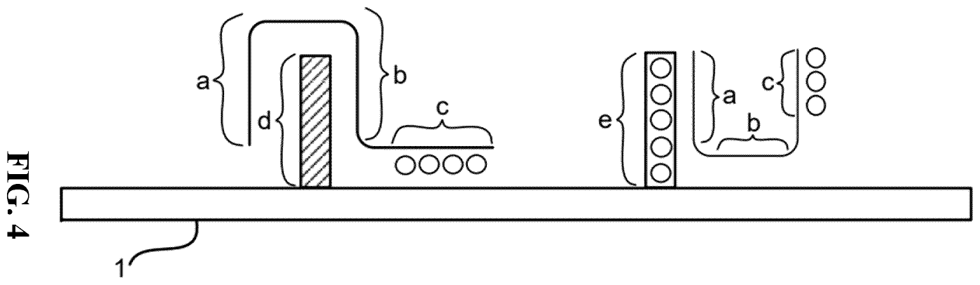

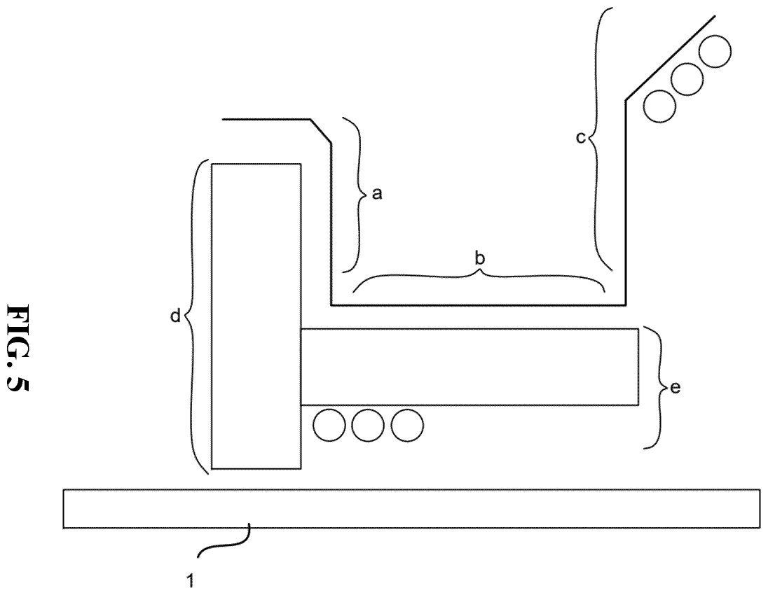

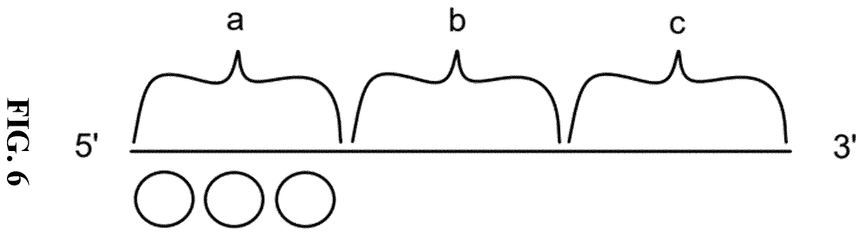

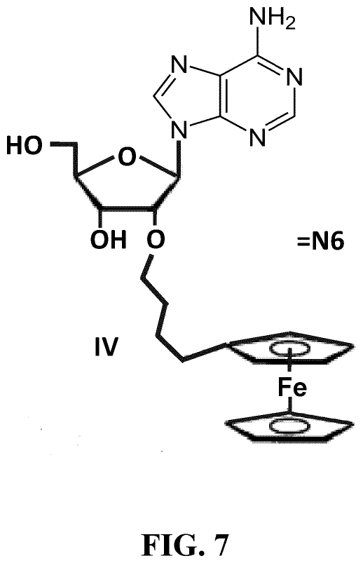

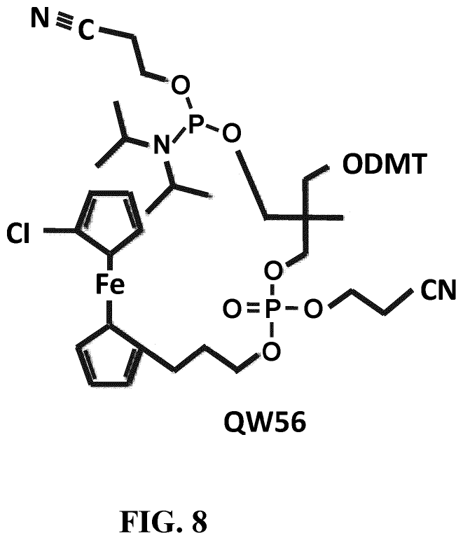

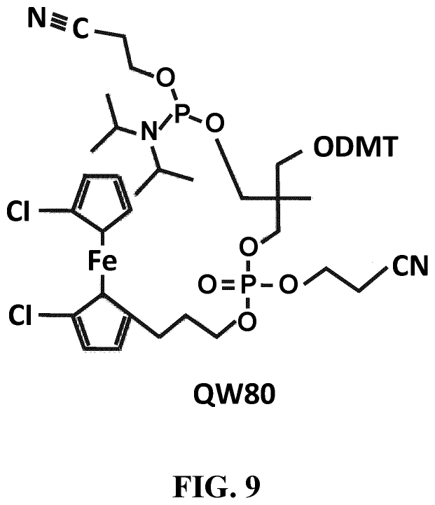

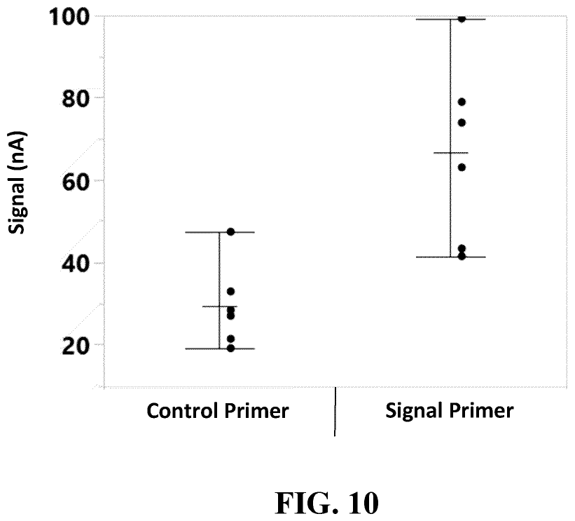

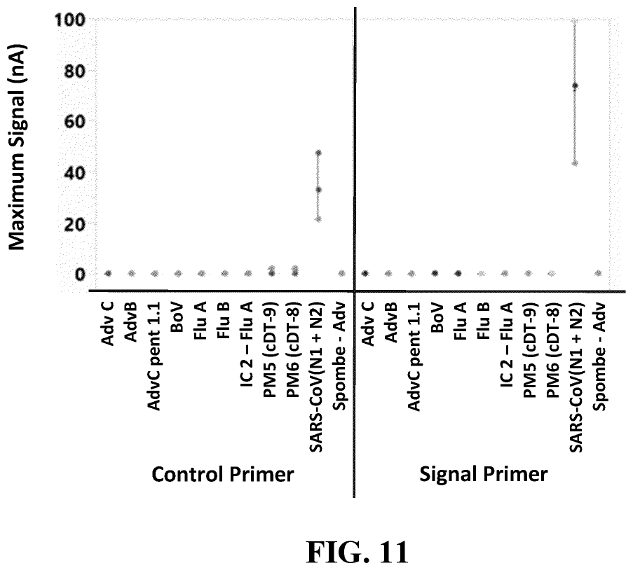

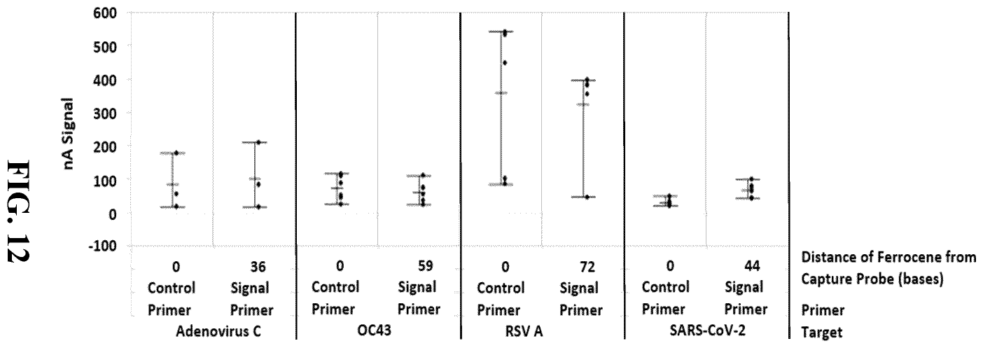

Alternatively, the primer, single stranded signal amplicon or double stranded signal amplicon may contain an extra terminal nucleoside at an end of the nucleic acid (n+1 or n+2), which are used to covalently attach the electron transfer moiety but which do not participate in base pair hybridization during PCR or detection. FIG. 2j shows an extra terminal nucleoside linking the signal moiety at the 5′-end of the double stranded signal amplicon and a second signal moiety is conjugated internally to the signal primer itself. FIG. 2k shows an extra terminal nucleoside linking the signal moiety at the 5′-end of the double stranded signal amplicon.

Alternatively, the primer may contain a linker, which is used to covalently attach the electron transfer moiety. FIG. 2l shows a linker on the 5′-end linking the signal moiety. FIG. 2m shows a linker on the 3′-end linking the signal moiety. FIG. 2n shows an embodiment where the primer contains a signal moiety at both the 5′-and 3′-ends.

FIG. 3 shows an embodiment of the system disclosed herein. As seen in FIG. 3, a capture probe bound to a detectable label (shown as a circle) can be individually or serially spotted onto the detection site. The capture probe bound to a detectable label can be specific for the analyte of interest. The capture probe bound to a detectable label can serve as a control and may contain its own signal that is distinguishable from that which is bound to the analyte. Stated another way, the labeled nucleic acid capture probes can be of the same or different redox potentials as the label on the single stranded signal amplicon. In some instances, the single stranded signal amplicon can bind to the labeled capture probe and in some instances it cannot bind. In some embodiments, the label on the capture probe and the label on the single stranded signal amplicon are identical, but they need not be. In some embodiments, they are energy transfer moieties. In some embodiments, they are ferrocene-based, ferrocene-derivative compounds, methylene blue or osmium.

FIG. 4 shows an embodiment of the system disclosed herein. Because the amplicon no longer needs to have a portion, which binds to a signal probe as in a typical sandwich assay, the amplicon can be shorter. Still, in some instances, it may be beneficial to amplify a longer amplicon and have multiple capture probes that bind different portions of the amplicon. The method can make use of one or more capture probes on the same detection electrode, which bind to different portions of a single stranded signal amplicon. In some embodiments, the portion of the single stranded signal amplicon (a) that binds a first capture probe (e) does not cross-hybridize with a second capture probe (d). In some embodiments, the portion of the single stranded signal amplicon (b) that binds a first capture probe (d) does not cross-hybridize with a second capture probe (e). In some embodiments, the first portion of the single stranded signal amplicon (b) binds a first capture probe (d) and does not cross-hybridize with a second capture probe (e), which binds a second portion of the single stranded signal amplicon (a). In some embodiments, the first portion of the single stranded signal amplicon (a) can bind a first capture probe (d) and a second capture probe (e), the second portion of the single stranded signal amplicon (b) cross-hybridizes with a second capture probe (e) and a first capture probe (d). In some embodiments, the first portion of the single stranded signal amplicon (a) can bind a first capture probe (d) and a second capture probe (e) but the second portion of the single stranded signal amplicon (b) can only bind with a second capture probe (e).

FIG. 5 shows an embodiment of the system disclosed herein. As seen in FIG. 5, in some embodiments, the single stranded signal amplicon has three portions. The first portion (a) is bound to a capture probe (d), the second portion (b) is bound to a signal probe (e) and the third portion is bound to an electron transfer moiety (c). In some embodiments, the single stranded signal amplicon detectable label has a different potential than the corresponding signal probe. In some embodiments, the single stranded signal amplicon detectable label has the same potential than the corresponding signal probe. In some embodiments, the portion of the single stranded signal amplicon (a) that binds the capture probe (d) does not cross-hybridize with the signal probe (e). In some embodiments, the portion of the single stranded signal amplicon (a) that binds the capture probe cross-hybridizes with the signal probe (b).

FIG. 6 shows an embodiment of the single stranded signal amplicon disclosed herein. As seen in FIG. 6, section c is the region of the single stranded signal amplicon that can bind to the capture probe. Section b is the primer region of the single stranded signal amplicon and it cannot bind to the capture probe. Section a is the region of the signaling moiety of the single stranded signal amplicon, which can also not bind to the capture probe. In some embodiments, section a is 5-100 base pairs away from section c. In some embodiments, section a is 5-200 base pairs away from section c. In some embodiments, section a is 25-100 base pairs away from section c.

FIG. 7 shows N6. N6 is a label that can be used; its synthesis is described in commonly owned U.S. Pat. No. 7,393,645, which is herein incorporated by reference in its entirety.

FIG. 8 shows QW56 ferrocene label. QW56 and QW80 ferrocene labels can be prepared using routine DNA synthesis techniques essentially as described in commonly owned application PCT/US08/82666 (published as WO/2009/061941A2 and U.S. Pat. No. 7,820,391), which are herein incorporated by reference in their entirety. In U.S. Pat. No. 9,891,215 (which is herein incorporated by reference in its entirety), FIG. 3A depicts QW 56 and FIG. 3B depicts QW80.

FIG. 9 shows QW80 ferrocene label.

FIG. 10 shows signal (nA) generated by SARS-COV-2 amplicons. Data from both detection pads for three replicates are shown.

FIG. 11 shows the maximum signal (nA) from all targets in the detection zone.

FIG. 12 compares the signal (nA) from targets using a traditional sandwich assay (control primer) and a signal primer for the targets shown.

### DETAILED DESCRIPTION

### Definitions

As used in the claims and herein, the following terms have the following definitions:

As used herein, “amplification” refers to any in vitro method for increasing the number of copies of a nucleotide sequence with the use of a polymerase. Nucleic acid amplification results in the incorporation of nucleotides into a nucleic acid molecule (e.g., DNA) or primer thereby forming a new nucleic acid molecule complementary to the nucleic acid template. The formed nucleic acid molecule and its template can be used as templates to synthesize additional nucleic acid molecules. As used herein, one amplification reaction may consist of many rounds of nucleic acid synthesis. Amplification reactions include, for example, polymerase chain reactions (PCR). One PCR reaction may consist of 5 to 100 “cycles” of denaturation and synthesis of a nucleic acid molecule. An “amplification primer” or “primer” is a primer for amplification of a target sequence by primer extension.

“Amplicon” is a nucleic acid molecule, which comprises a primer or a portion of a primer and a newly synthesized strand, which is the complement of the sequence downstream of the primer binding site. Extension products result from hybridization of a primer to a template containing a complementary sequence and extension of the primer by polymerase using the template.

By “analyzing” is meant measuring, detecting or determining the presence, absence or composition of something.

An “analyte” is anything that can selectively bind a capture binding ligand. Analytes may be natural, biological or synthetic, e.g., as in any of synthetic or other molecules used for drug discovery that manifest unusually good or specific binding affinity to a “capture binding ligand.” Both analytes and capture binding ligands may consist of one or more different domains. The person of skill will appreciate that complementary orientations between the analyte and capture binding ligands are necessary. Suitable analytes include organic and inorganic molecules, including biomolecules. In an embodiment, the analyte may be an environmental pollutant (including pesticides, insecticides, toxins, etc.); a chemical (including solvents, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic (such as pathogenic bacteria) and eukaryotic cells, including mammalian tumor cells); viruses (including retroviruses, herpesviruses, adenoviruses, lentviruses, etc.); and spores; etc.

Suitable nucleic acid target analytes include, but are not limited to, the nucleic acid of any number of viruses including orthomyxoviruses, (e.g., influenza virus), paramyxoviruses (e.g respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and-II), papovaviruses (e.g., papillomavirus), polyomaviruses, and picomaviruses, and the like), and bacteria (including a wide variety of pathogenic and non-pathogenic prokaryotes of interest including Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprae; Clostridium, e.g., C. botulinum, C. tetani, C. dificile, C. perfringens; Comyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lamblia Y. pestis, Pseudomonas, e.g., P. aeruginosa, P. putida; Chlamydia, e.g., C. trachomatis; Bordetella, e.g., B. pertussis; Treponema, e.g., T. palladium; and the like) (collectively “Bacterial and Viral Targets”).

Suitable nucleic acid target analytes include, but are not limited to, the nucleic acid of any number of gram-positive organisms including, Bacillus cereus group, Bacillus subtilis group, Corynebacterium, Cutibacterium acnes, Propionibacterium acnes, Enterococcus, Enterococcus faecalis, Enterococcus faecium, Lactobacillus, Listeria, Listeria monocytogenes, Micrococcus, Staphylococcus, Staphylococcus aureus, Staphylococcus epidermidis, Staphylococcus lugdunensis, Streptococcus, Streptococcus agalactiae (GBS), Streptococcus anginosus group, Streptococcus pneumoniae, Streptococcus pyogenes (GAS), Resistance Genes, mecA, mecC, vanA, or vanB (collectively “Gram Positive Targets”).

Suitable nucleic acid target analytes include, but are not limited to, the nucleic acid of any number of gram-negative organisms including, Acinetobacter baumannii, Bacteroides fragilis, Citrobacter, Cronobacter sakazakii, Enterobacter (non-cloacae complex), Enterobacter cloacae complex, Escherichia coli, Fusobacterium nucleatum, Fusobacterium necrophorum, Haemophilus influenzae, Klebsiella oxytoca, Klebsiella pneumoniae group, Morganella morganii, Neisseria meningitidis, Proteus, Proteus mirabilis, Pseudomonas aeruginosa, Salmonella, Serratia, Serratia marcescens, Stenotrophomonas maltophilia, Resistance Genes, CTX-M, IMP, KPC, NDM, OXA (OXA-23 and OXA-48), or VIM (collectively “Gram Negative Targets”).

Suitable nucleic acid target analytes include, but are not limited to, the nucleic acid of any number of fungal organisms including, Candida albicans, Candida auris, Candida dubliniensis, Candida famata, Candida glabrata, Candida guilliermondii, Candida kefyr, Candida krusei, Candida lusitaniae, Candida parapsilosis, Candida tropicalis, Cryptococcus gattii, Cryptococcus nehoformans, Fusarium, or Rhodotorula (collectively “Fungal Targets”).

In some embodiments the targets are human-specific infectious disease agents or targets, with the markers or targets being nucleic acid markers.

By “array” is meant a plurality of distinct sites bearing different capture binding ligands. In some embodiments, the array is “addressable” insofar as the individual sites have a predetermined or determinable location relative to one another, optionally with the help of electronic connectors and/or software.

A “capture binding ligand” is synonymous with a “capture probe” or “capture binding probe” and is a compound that exhibits a relatively strong or specific affinity for another compound such that it is capable of abstracting that compound away from a group of other compounds in a mixture of compounds. The capture binding ligand may be a protein, carbohydrate, nucleic acid, small molecule, or any combination of these.

By “double stranded signal amplicon” is meant a double stranded amplicon created by use of a primer during PCR comprising an electrochemically detectable label. The Double Stranded Signal Amplicon comprises nucleic acids and a signal primer.

By “electrode” is meant a composition, which, when connected to an electronic device, is able to sense a current or charge and convert it to a signal. Electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo206), tungsten oxide (W03) and ruthenium oxides; and carbon (including glassy carbon electrodes, graphite and carbon paste).

“Electrochemical detection” means the use of at least two electrodes to apply potential and measure current produced by a chemical reaction. For the sake of clarity, “Electrochemical Detection” excludes detection (i) of conductivity, impedance or capacitance of a droplet, a portion of a droplet, or the contents of a droplet; (ii) by electrochemilumenescence; and (iii) by optical means.

The terms “electron donor moiety”, “electron acceptor moiety”, and “electron transfer moieties” or grammatical equivalents herein refers to molecules capable of electron transfer under certain conditions. It is to be understood that electron donor and acceptor capabilities are relative; that is, a molecule, which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds and selection of those compounds is within the skill of the skilled artisan. One advantage of redox-mediated electronic detection is that there is a variety of different electronic transfer moiety labels each having its own distinct potential that can be selectively measured or filtered. Some electron transfer moieties include, but are not limited to, transition metal complexes, organic electron transfer moieties, electrodes, metallocenes such as ferrocene, and ferrocene derivatives or methylene blue or osmium.

As used herein, the terms “hybridization” and “hybridizing” refer to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well-known in the art, are used.

The term “immobilize” or derivative terms thereof, includes affixation, association or binding, whether covalently or non-covalently.

By “infectious disease” is meant a disease marked by an infectious disease marker, agent or target, whether it be viral, bacterial or fungal. Illustrative infectious diseases targets include, e.g., natural, synthetic or amplified biomolecules such as: Viral Targets, Gram Negative Targets, Gram Positive Targets and Fungal Targets.

By “label” is meant something that can signal or be stimulated to signal an event or the presence of a molecule or complex of molecules. Labels may include, e.g., dyes, radioactive atoms or molecules, redox-active compounds, enzymes, enzyme substrates, nucleic acids, derivatives thereof the like. Redox-active labels come in a variety of different potentials that can be used, similar to the existence of different color dyes and chemilumiscent compounds.

By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer.

By “nucleic acid” or “oligonucleotide” or grammatical equivalents herein means at least two nucleotides covalently linked together. Nucleic acids generally contain phosphodiester bonds, although in some cases, as outlined below, nucleic acid analogs are included that may have alternate backbones, comprising, for example, phosphoramide. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occuring analog structures. Thus, for example, the individual units of a peptide nucleic acid, each containing a base, are referred to herein as a nucleoside.

As used herein “nucleotide” refers to a base-sugar-phosphate combination. Nucleotides are monomeric units of a nucleic acid sequence (DNA and RNA). The term nucleotide includes mono-, di- and triphosphate forms of deoxyribonucleosides and ribonucleosides and their derivatives. The term nucleotide particularly includes deoxyribonucleoside triphosphates such as dATP, dCTP, dITP, dUTP, dGTP, dTTP, or derivatives thereof. Such derivatives include, for example, [αS] dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddITP. As used herein, nucleotides are unlabeled.

As used herein “polymerase” refers to any enzyme having a nucleotide polymerizing activity. Polymerases (including DNA polymerases and RNA polymerases) include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furious (Pflu) DNA polymerase, DEEPVENT DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME) DNA polymerase, Methanobacterium thermoautotrophicum (Mth) DNA polymerase, mycobacterium DNA polymerase (Mtb. Mlep), and mutants, variants and derivatives thereof. RNA polymerases such as T3, T5 and SP6 and mutants, variants and derivatives thereof may also be used.

As used herein, “primer” refers to a synthetic or biologically produced single-stranded oligonucleotide that is extended by covalent bonding of nucleotide monomers during amplification or polymerization of a nucleic acid molecule. Nucleic acid amplification often is based on nucleic acid synthesis by a nucleic acid polymerase or reverse transcriptase. Many such polymerases or reverse transcriptases require the presence of a primer that can be extended to initiate such nucleic acid synthesis.

As used herein, “probe” refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences. By “redox-active” compound or moiety is meant one capable of transferring, shuttling or receiving electrons from another redox-active compound. Some redox-active compounds include electrodes and metallocenes, including ferrocenes and derivatives thereof, methylene blue or osmium.

The “sample solution” may comprise any number of things, including, but not limited to, bodily fluids (including, but not limited to, blood, urine, serum, lymph, saliva, anal and vaginal secretions, perspiration and semen, of virtually any organism, such as mammalian samples including human samples); environmental samples (including, but not limited to, air, agricultural, water and soil samples); biological warfare agent samples; research samples (i.e., in the case of nucleic acids, the sample may be the products of an amplification reaction, including both target and signal amplification such as PCR amplification reaction); purified samples, such as purified genomic

DNA, RNA, proteins, etc.; raw samples (bacteria, virus, genomic DNA), etc.; as will be appreciated by those in the art, virtually any experimental manipulation may have been done on the sample.

By “signal primer” is meant a primer comprising an electrochemically detectable label.

By “signal probe” is meant a probe molecule that bears a label of some sort that can bind to and signal the presence of analyte. For electrochemical detection, the label is often ferrocene or a ferrocene-derivative, which bind to one domain of analyte while another domain of the analyte binds to the capture binding ligand on a solid support surface site (configurations known as “sandwich assays”).

By “single stranded signal amplicon” is meant an amplicon created by use of a primer during PCR comprising an electrochemically detectable label. The single stranded signal amplicon comprises nucleic acids and a signal primer.

A “solid support” or “support” may be any material or matrix suitable for attaching oligonucleotides/capture probes. Such oligonucleotides and/or capture probes may be added or bound (covalently or non-covalently) to the supports by any technique or any combination of techniques well known in the art. Supports may be anything other than an aqueous phase at room temperature and include, e.g., beads, gels, columns, column matrices, multi-titer plates, paper, membranes, printed circuit boards, or other array surfaces or supports.

The term “target nucleic acid” or “target” or grammatical equivalents herein refer to nucleic acid sequences to be amplified or detected. These include the original nucleic acid sequence to be amplified, its complementary second strand and either strand of a copy of the original sequence, which is produced by replication or amplification. A target sequence may also be referred to as a template for extension of hybridized primers.

The target sequence may be a portion of a gene, a regulatory sequence, genomic DNA, cDNA, RNA including mRNA and rRNA, or others. It may be any length, with the understanding that longer sequences are more specific. As will be appreciated by those in the art that the complementary target sequence may take many forms. For example, it may be contained within a larger nucleic acid sequence, i.e., all or part of a gene or mRNA, a restriction fragment of a plasmid or genomic DNA, among others. As is outlined more fully below, probes are made to hybridize to target sequences to determine the presence or absence of the target sequence in a sample. The target sequence may also be comprised of different target domains; for example, a first target domain of the sample target sequence may hybridize to a capture probe or a portion of a capture probe, a second target domain may hybridize to a portion of a different capture probe. The target domains may be adjacent or separated. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. A target refers to a nucleic acid molecule to which a particular primer or probe is capable of preferentially hybridizing.

As used herein, “target sequence” refers to a nucleic acid sequence within the target molecules to which a particular primer or probe is capable of preferentially hybridizing.

The term “template” as used herein refers to a double-stranded or single-stranded molecule, which is to be amplified, synthesized or sequenced. In the case of a double-stranded DNA molecule, denaturation of its strands to form a first and a second strand is performed to amplify, sequence or synthesize these molecules. A primer, complementary to a portion of a template is hybridized under appropriate conditions and the polymerase (DNA polymerase or reverse transcriptase) may then synthesize a nucleic acid molecule complementary to said template or a portion thereof. The newly synthesized molecule may be equal or shorter in length than the original template. Mismatch incorporation during the synthesis or extension of the newly synthesized molecule may result in one or a number of mismatched base pairs. Thus, the synthesized molecule need not be exactly complementary to the template. The template can be an RNA molecule, a DNA molecule or an RNA/DNA hybrid molecule. A newly synthesized molecule may serve as a template for subsequent nucleic acid synthesis or amplification.

Other terms used in the fields of recombinant DNA technology and molecular and cell biology as used herein will be generally understood by one of ordinary skill in the applicable arts.

### General Description of Devices and Methods

Devices and methods for nucleic acid amplification and detection are disclosed. The devices may be equipped for electrochemical detection methods. Disclosed are methods and reagents for the amplification of nucleic acid sequences and their use in the detection of nucleic acids.

In a first embodiment, an oligonucleotide primer (i.e., signal primer) for use in a nucleic acid amplification assay is disclosed comprising a primer region and an electron transfer moiety (ETM) (e.g., a ferrocene label). In some embodiments, the primer does not comprise a secondary structure in solution at any temperature. In some embodiments, the primer is capable of binding a target nucleic acid at any temperature. In some embodiments, the oligonucleotide primer is self-annealing.

Thus, in a first aspect, methods of amplifying one or more nucleic acid molecules are provided, comprising: (a) mixing one or more templates or target nucleic acid molecules or sample suspected to comprise a target nucleic acid with one or more signal primers described herein and the reagents necessary to carry out amplification; and (b) incubating said mixture under conditions sufficient to amplify one or more nucleic acid molecules complementary to all or a portion of said templates or target molecules. The amplified nucleic acid molecules comprise one or more signal primers disclosed herein or portions thereof. In one aspect, an electron transfer moiety (ETM) is incorporated at or near one or both termini of the synthesized or amplified nucleic acid molecules produced by the amplification. In one aspect, the electron transfer moiety (ETM) is incorporated at or near the 5′-end.

In another aspect, the method comprises a method of performing a nucleic acid amplification assay comprising: (a) combining reagents for nucleic acid amplification, nucleic acid polymerase, a target nucleic acid or a sample suspected to comprise a target nucleic acid, and a modified primer, said modified primer comprising a detectable label wherein the detectable label is ferrocene; (b) cycling the mixture of (a) to provide multiple copies of an amplicon incorporating said modified primer if target nucleic acid is present in the sample; (c) denaturing the amplicon to a single strand signal amplicon; (d) exposing said single stranded signal amplicon to a capture oligonucleotide complimentary to said single strand signal amplicon; (e) hybridizing a hybridization region of said single stranded signal amplicon with said capture oligonucleotide, and (f) detecting said label associated with said hybridization. In some embodiments, the detection is electrochemical detection. In some embodiments, the target nucleic acid is a DNA. In some embodiments, the nucleic acid is an RNA and the method further comprises the step of reverse transcribing RNA to generate cDNA using a reverse transcriptase. In some embodiments, the nucleic acid polymerase is a DNA polymerase.

In yet another embodiment, the method comprises a method of performing a nucleic acid amplification assay comprising: (a) combining reagents, nucleic acid polymerase, a target nucleic acid or a sample suspected to comprise a target nucleic acid, a modified primer and a second primer, said modified primer comprising a detectable label, wherein the detectable label is ferrocene, said second primer comprising a sequence of bases complimentary to a second region of said target nucleic acid and not having a detectable label; (b) cycling the mixture of (a) to provide multiple copies of a double stranded amplicon incorporating said modified primer and said second primer if target nucleic acid is present in the sample; (c) denaturing the amplicons incorporating said modified primer and denaturing the amplicon incorporating said second primer to a single strand; (d) exposing the single stranded signal amplicon incorporating said modified primer to a capture oligonucleotide complimentary to said single stranded signal amplicon and exposing the second single stranded amplicon to a capture oligonucleotide complimentary to said second single stranded amplicon; (e) hybridizing said single stranded signal amplicon incorporating said modified primer, with said capture oligonucleotide and hybridizing said second single stranded amplicon, with said capture oligonucleotide; and (f) detecting said label associated with said first primer incorporated into said amplicon. In some embodiments, the detection is electrochemical detection. In some embodiments, the target nucleic acid is a DNA. In some embodiments, the nucleic acid is an RNA and the method further comprises the step of reverse transcribing RNA to generate cDNA using a reverse transcriptase. In some embodiments, the nucleic acid polymerase is a DNA polymerase.

In yet another aspect, the method comprises a method of performing a nucleic acid amplification assay comprising: (a) combining reagents, polymerase, a target nucleic acid or a sample suspected to comprise a target nucleic acid, a first modified primer and a second modified primer, said first modified primer comprising a first detectable label and said second modified primer comprising a second detectable label, wherein the first and second detectable label is ferrocene, said second modified primer comprising a sequence of bases complimentary to a second region of said target nucleic acid; (b) cycling the mixture of (a) to provide multiple copies of a double stranded amplicon incorporating said first modified primer and said second modified primer if target nucleic acid is present in the sample; (c) denaturing the amplicons incorporating said first modified primer and denaturing the amplicon incorporating said second modified primer to a single strand; (d) exposing the single stranded signal amplicon incorporating said first modified primer to a capture oligonucleotide complimentary to said first single stranded signal amplicon and exposing the second single stranded signal amplicon to a capture oligonucleotide complimentary to said second single stranded signal amplicon; (e) hybridizing said first single stranded signal amplicon incorporating said first modified primer, with said first capture oligonucleotide and hybridizing said second single stranded signal amplicon, with said second capture oligonucleotide; and (f) detecting said label associated with said first primer incorporated into said amplicon and detecting said label associated with said second primer incorporated into said amplicon. In some embodiments, the detection is electrochemical detection. In some embodiments, the target nucleic acid is a DNA. In some embodiments, the nucleic acid is an RNA and the method further comprises the step of reverse transcribing RNA to generate cDNA using a reverse transcriptase. In some embodiments, the polymerase is a DNA polymerase.

### System Overview

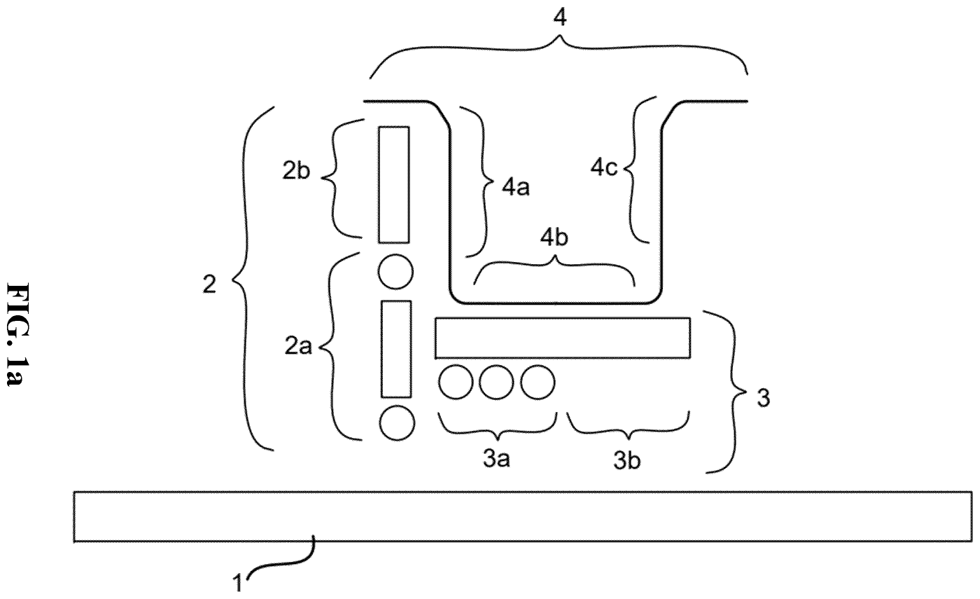

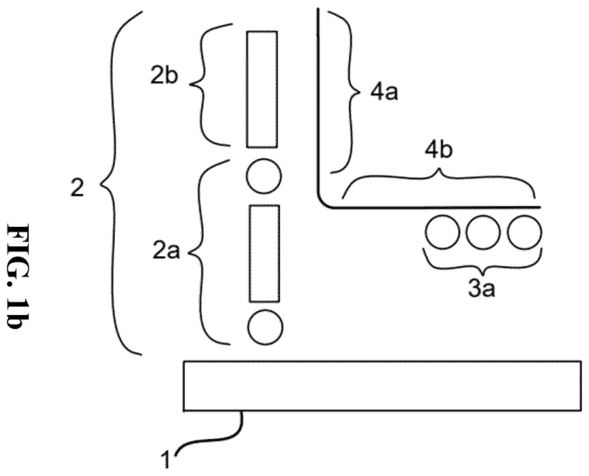

Current electrochemical detection assays are based on the principles of nucleic acid hybridization using a sandwich assay format, wherein a single-stranded amplicon binds to a sequence-specific signal probe and the signal probe/single-stranded amplicon complex (“signal probe complex”) binds to an electrode-bound capture probe as set forth in FIG. 1a, which is a schematic diagram showing signal generation using the current capture probe/signal probe sandwich technology. In contrast thereto, provided herein are methods and systems, which remove the need for a signal probe and a signal probe complex. The disclosed methods and systems find utility in genotyping, subtyping, isotyping or expression assays.

To begin, target nucleic acids are extracted from samples and amplified using Polymerase Chain Reaction (PCR) using a signal primer to form double-stranded signal DNA. In some embodiments, the target nucleic acid is a DNA, which is directly amplified using a primer pair comprising at least one signal primer. In some embodiments, the target nucleic acid may be an RNA, which is subjected to a reverse transcription amplification prior to amplification using Polymerase Chain Reaction (PCR) using a signal primer to form double-stranded signal DNA. Herein, the primer for reverse transcription may be an additional reverse transcription primer or may be the signal primer. In some instances, the reverse transcription and amplification reaction may be performed in a one-step reaction (RT-amplification). The resulting double-stranded signal DNA is then digested by an exonuclease to create a single-stranded signal DNA. Binding of the single-stranded signal DNA with the capture probe brings the electron transfer moiety, ETM, (e.g., a ferrocene label) near the surface of the gold electrode. An electric signal, specific to the hybridized complex, is generated as a byproduct of a reduction-oxidation reaction when voltage is applied to the system (FIG. 1b).

Sequential analysis of each electrode allows detection of multiple analyte targets. The detection instrument measures and interprets this electrical output (in nanoamperes, nA) to determine the results for each target nucleic acid (e.g., in a qualitative fashion as “Detected” or “Not Detected”). Herein, at least one “signal primer” as provided herein is used for amplification of the target nucleic acid. In some embodiments, the signal primers are designed to have a ferrocene label (such as, e.g., N6, QW56 or QW80) at the 5′-end or close to the 5′-end (e.g., attached to any one nucleotide of nucleotides 1 to 3 at the 5′-end or attached to any one nucleotide of nucleotides 1 to 2 at the 5′-end) of the non-phosphorylated oligonucleotide primers. In some embodiments, the amplicon generated by using a signal primer has a ferrocene label on its 5′-end or close to its 5′-end (e.g., within nucleotides 1 to 3 or nucleotides 1 to 2 of its 5′-end). As shown in FIG. 1b, the single stranded signal amplicon will bind to a capture probe and bring the ferrocene label near the surface of the gold electrode to generate signal when a voltage is applied. Therefore, signal probes will no longer be needed for detection.

Prior to this disclosure, it was unknown if nucleic acid amplification could occur using a primer linked to a ferrocene label. For example, it was not known whether the polymerase would recognize the primer with an ETM attached thereto (such as, e.g., a ferrocene label). It was also not known whether the ETM (such as, e.g., a ferrocene label) bound to the primer could withstand thermocycling. For example, it was not known whether the ETM (such as, e.g., a ferrocene label) itself could withstand thermocycling. It was also not known whether Taq polymerase or reverse transcriptase would be inhibited by the ETM (such as, e.g., a ferrocene label). Moreover, prior to this disclosure it was unknown if signal detection could occur using a primer linked to an ETM (such as, e.g., a ferrocene label) in that it was unknown whether the detectable label could be held close enough to the electrode to detect a signal.

For example, in traditional prior art sandwich assays, an ETM (such as, e.g., a ferrocene label) is attached to the signal probe and the signal probe binds to the amplicon. Herein, the gap between the signal probe binding site and the capture probe binding site commonly is zero bases. Alternatively, the sequence gap between the signal probe and capture probes may be zero to two bases. Moreover, it is known for traditional sandwich assay systems that the signal is drastically reduced if the ETM (such as a ferrocene label) is 5 or more bases away from the capture probe.

If the ferrocene label is attached to the 5′-end or close to the 5′-end (e.g., within nucleotides 1 to 3 or nucleotides 1 to 2 of its 5′-end) of an amplicon as in the some embodiments of the instant disclosure, it will be further away from the capture probe (in some cases as much as 100 base pairs away) and thus, a skilled artisan would not know whether such a “far away” ferrocene label would be able to generate a detectable signal. Accordingly, a capture probe signal primer complex is provided herein, in which the ETM (such as, e.g., a ferrocene label) is greater than 5 base pairs away from the position to which the capture probe hybridizes to. In some embodiments, a capture probe signal primer complex is provided in which the ETM (such as, e.g., a ferrocene label) is greater than 10 base pairs away from the position to which the capture probe hybridizes to. In further embodiments, a capture probe signal primer complex is provided herein, in which the ETM (such as, e.g., a ferrocene label) is greater than 50 base pairs away from the position to which the capture probe hybridizes to. In further embodiments, a capture probe signal primer complex is provided herein, in which the ETM (such as, e.g., a ferrocene label) is greater than 100 base pairs away from the position to which the capture probe hybridizes to. In further embodiments, a capture probe signal primer complex is provided herein, in which the ETM (such as, e.g., a ferrocene label) is greater than 200 base pairs away from the position to which the capture probe hybridizes to. In some embodiments, a capture probe signal primer complex is provided herein, in which the ETM (such as, e.g., a ferrocene label) is between 5-100 base pairs away from the position to which the capture probe hybridizes to. In additional embodiments, a capture probe signal primer complex is provided herein, in which the ETM (such as, e.g., a ferrocene label) is between 5-500 base pairs away from the position to which the capture probe hybridizes to. In certain embodiments, a capture probe signal primer complex is provided herein, in which the ETM (such as, e.g., a ferrocene label) is between 5-50 base pairs away from the position to which the capture probe hybridizes to.

### Electrochemical Detection Systems

Further provided herein is an electrochemical detection system, which specifically detects target nucleic acid segments. The system utilizes capture probes such as nucleic acid or peptide nucleic acid probes, which are complementary to and specifically hybridize with target nucleic acid segments bound to a detectable label in order to generate a measurable current, when an amperometric potential is applied. In some embodiments, the electrochemical signal is proportional to the quantity of target nucleic acid in a sample.

Also provided are compositions and methods useful in detecting target analytes in a sample. Signal primers, single stranded signal amplicons and hybridization complexes bound to single stranded signal amplicons provide a new and useful way to detect target analytes. Herein, the signal primer used for amplification of the target nucleic acid comprises a nucleic acid capable of specifically hybridizing to the target nucleic acid and an electrochemically detectable signaling moiety. Hence, after amplification using the signal primer the single stranded signal amplicon comprises nucleic acid and an electrochemically detectable signaling moiety. The assay complex comprises a capture binding ligand attached to an electrode (e.g., a gold electrode) and single stranded signal amplicon bound to the capture binding ligand, wherein the single stranded signal amplicon comprises nucleic acid and an electrochemically detectable signaling moiety.

### Signal Primers Simplify Assay Development

An electrochemical detection system utilizing signal primers is described herein. Traditional prior art electrochemical detection systems utilize a sandwich assay for detection. As such, an amplicon must be long enough to bind to both, a capture probe and a signal probe. In contrast thereto, using a signal primer as disclosed herein reduces the length of the amplicon because the need to bind a signal probe is eliminated. This makes the design of primers easier because (1) a shorter region to distinguish the target nucleic acid is needed; and (2) unlike in traditional sandwich assays, where four conserved regions are needed (forward primer, reverse primer, signal probe and capture probe), with a signal primer as disclosed herein only three conserved regions are needed (forward primer, reverse primer, and capture probe). Furthermore, employing an electrochemical detection moiety bound to a nucleic acid amplicon eliminates the need to reduce background signal or to enhance signal to noise ratio. Specifically, there is no need to include a donor/quencher dye pair, i.e., a fluorescent donor dye or a quencher for the donor fluorophore.

### Signal Primers Improve Sensitivity

Improved sensitivity has been observed using signal primers. Without being bound to any particular theory, in prior art sandwich assays, if the amplicon does not bind to the signal probe with 100% efficiency, then the sensitivity of the assay is negatively impacted. By eliminating the requirement for the amplicon to bind a signal probe, the sensitivity of the assay can be improved. It is also believed that the inclusion of an ETM (such as, e.g., a ferrocene label), while not impeding amplification, makes the signal primer less capable of mis-priming to the target nucleic acid molecule. The increase in sensitivity is particularly important for the amplification of template nucleic acids that are difficult to amplify and that produce low amounts or none of the desired amplification product in PCR reactions.

### Signal Primers Reduce the Time to Result

Recent studies have shown that patients with severe sepsis or septic shock showed an increased likelihood of death of 7.6% for every hour in which antibiotic therapy is not applied. Liang et al., Empiric Antimicrobial Therapy in Severe Sepsis and Septic Shock: Optimizing Pathogen Clearance, Curr Infect Dis Rep. 2015 July; 17(7):493. Survival rates could increase if detection systems performed quicker.

As laid out above, when using a signal primer for amplification the amplicon does not require a signal probe binding region and consequently, the amplicon can be shorter. A shorter amplicon means the time needed for amplification is reduced. Reducing the amplification time, reduces the time to result, which in turn can save a patient's life.

Additionally, in prior art sandwich assays, the amplicon must first bind to a signal probe during a hybridization reaction and then the signal probe-amplicon complex binds to the capture probe. The use of a signal primer eliminates the need for a separate signal probe/amplicon hybridization step, further reducing the time to result.

### Signal Primers Simplify Manufacturing Processes

Eliminating the need for signal probes simplifies the detection hybridization complex from three components to two, which in turn simplifies the manufacturing process for the diagnostic test/kit.

### Distinction Over the Prior Art

Certain types of signal primers are known in the art (see e.g., U.S. Pat. Nos. 8,323,929, 9,068,948 and U.S. Publication No. 20190024167). However, these prior art signal primers all utilize fluorescence labels. In contrast thereto, signaling primers provided herein use a label that can be detected by electrochemical detection. This difference is significant because, prior to this disclosure it was unknown if nucleic acids (e.g., DNA) could be amplified in the presence of an ETM (such as, e.g., a ferrocene label). For example, it was not known whether Taq polymerase or reverse transcriptase would be inhibited by an ETM (such as, e.g., a ferrocene label). More importantly, it was not known if a single stranded signal amplicon could be detected when bound to a capture probe. This is due to the distance of the ETM (such as, e.g., a ferrocene label) from capture probe. In traditional sandwich assay systems, as the signal probe moves away from capture probe, signal is reduced drastically.

The present embodiments do not use a reporter probe, the 3′ end of which hybridizes to the complement of the 5′ adapter sequence of the signal primer to produce a 5′ overhang as in disclosed in U.S. Pat. No. 8,323,929. Moreover, the present embodiments also do not use polymerase to fill in the overhang and synthesize the complement of the 5′ overhang of the reporter probe. The present embodiments do not detect synthesis of the reporter probe complement, either directly or indirectly, as an indication of the presence of the target nucleic acid.

Further, the present embodiments do not detect the presence or quantity of nucleic acid of interest by detecting energy transfer between a first energy transfer element in a nucleic acid primer and a second energy transfer element in an incorporated labeled nucleotide as disclosed in U.S. Pat. No. 9,068,948. The present embodiments do not detect energy transfer between labeled primers, energy transfer between a labeled primer and nucleotide(s), energy transfer between labeled nucleotides, energy transfer between a fluorescent intercalator and a labeled primer or nucleotide(s), or energy transfer between a labeled probe and nucleotide(s). The present embodiments do not depend upon the proximity between the primers or nucleic acid constructs on each strand as in U.S. Pat. No. 9,068,948.

Further, the signal primers of the present embodiments are not immobilized as in U.S. Publication No. 20190024167. In the present embodiments, there is no need to reduce or quench energy emitted or other detectable properties from the labels as in U.S. Publication No. 20190024167.

The present system does not allow detection in real-time. Unlike fluorescent-based systems, in which the label may be monitored as the reaction is occurring, i.e., in “real-time,” the disclosed signal primers must bind to a capture probe for detection at the end of amplification. This is a so-called “end-point” detection system. As such, the system cannot be used to provide semi-quantitative or quantitative information about the initial amount of target present. Nevertheless, it is expected that signal generation is specific and proportional to the presence of appropriate target molecules in the sample. Further, the signal primers provided herein require hybridization to a capture probe for detection and cannot be detected directly from the PCR product.

### General Background on the Value of Nucleic Acid Testing

Applications of nucleic acid testing are broad. The majority of current commercial testing relates to infectious diseases including Chlamydia, gonorrhea, hepatitis and human immunodeficiency virus (HIV) viral load; genetic diseases including cystic fibrosis; coagulation and hematology factors including hemochromatosis; and cancer including genes for breast cancer. Other areas of interest include cardiovascular diseases and drug resistance screening, termed pharmacogenomics. The majority of testing currently occurs in centralized laboratories, which process hundreds if not thousands of samples a day.

Sequence-specific hybridization of labeled oligonucleotide probes has long been used as a means for detecting and identifying selected nucleotide sequences. Conventional detection methods for the final step in a nucleic acid analysis are well known in the art and include sandwich-type capture methods based on radioactivity, colorimetry, fluorescence, fluorescence resonance energy transfer (FRET) and electrochemistry.

Labeling probes with ferrocene has provided a relatively sensitive means for facilitating detection of probe hybridization. Several patents address electrochemical detection of nucleic acids, for example U.S. Pat. No. 10,001,476 (which is incorporated by reference in its entirety) discloses detection with capture probe controls, and various capture and signal probe configurations and combinations of configurations that can facilitate accurate and efficient multiplex analyte detection. U.S. Pat. No. 10,001,476 discloses single stranded DNA on an electrode (capture probe) that binds to a target. Such systems require that the ferrocene label be held in close proximity to the electrode to work. In order to achieve the proper orientation of the ferrocene label to the electrode, sandwich assays have been used in the prior art. As discussed above, sandwich assays require an amplicon capable of binding to both a capture and signal probe. Developing primers to amplify a unique, conserved sequence capable of binding both a capture and signal probe is complicated. As such, there is a need to develop a simpler system.

### Electrochemical Detection

The methods and systems provided herein include forming hybridization complexes on detection electrodes under conditions, wherein the single stranded signal amplicon specific for the nucleic acid capture probe is held in close proximity to the detection electrode and can be detected. Hence, methods provided herein include testing for the presence of the hybridization complex on the electrode under conditions, wherein the single stranded signal amplicon bound to the capture probe is in close proximity to the electrode and transfers electrons thereto. Single stranded signal amplicons and signal primers, which are not bound to the capture probe, are driven away from the electrode when the voltage/current is turned on and are not detected.

In some embodiments, the method of detection is implemented in a single-use cartridge using electrochemical detection. In some embodiments, electrochemical detection comprises electrodes comprising a monolayer comprising conductive oligomers, and a capture binding ligand.

In some embodiments, the method can make use of one or more capture probes on the same detection electrode, which bind to different portions of a single stranded signal amplicon. When multiple capture probes are used, each is specific for a different portion of the common nucleic acid sequence of interest. The nucleic acid sequence of interest can be amplified nucleic acid, e.g., through PCR. In some embodiments, the electrodes have a self assembling monolayer (“SAM”). In some embodiments, the electrodes have a mixed SAM of two or more species, each species featuring different chain lengths, conjugated bond numbers (if any) and/or substituents (if any). In some embodiments, the ETM is a detectable label, such as a ferrocene label. In some embodiments, the ferrocene label is selected from the group consisting of N6, QW56 and QW80. In certain embodiments, the detectable label is N6 (see, e.g., FIG. 7). In certain embodiments, the detectable label is QW56 (see, e.g., FIG. 8). In certain embodiments, the detectable label is QW80 (see, e.g., FIG. 9). Herein, the detection method is electrochemical.

In certain embodiments, the capture oligonucleotide is immobilized on a gold surface. In other embodiments, the capture oligonucleotide is immobilized on an electrode. In some embodiments, the single stranded signal amplicon comprising the modified signal primer hybridizes to the capture oligonucleotide.In some embodiments, the labels that are used are electron transfer moieties (ETMs) and the addressable solid support detection sites are detection electrodes spotted with capture probes. In some embodiments, e.g., nucleic acid analyte electrochemical detection embodiments employing detection electrodes, the sites also feature an insulating self-assembled monolayer or mixed monolayer.

Electrochemical detection is known to skilled artisans. Generally, at least a first input signal is applied to the assay complex and an output signal is received. The output signal is then processed to detect the presence of said target analytes. Some embodiments utilize a plurality of assay complexes each attached to a different cell or pad of the array.

### Hybridization

In the PCR reaction described above, amplicons are generated by two amplification primers, which generate two different single stranded regions. After amplification, each amplified single stranded signal amplicon can bind to a capture probe and produce a signal.

### Electron Transfer

In some embodiments, detection of an ETM is based on electron transfer through the stacked x-orbitals of double stranded nucleic acid. This basic mechanism is described in U.S. Pat. Nos. 5,591,578, 5,770,369, 5,705,348, and PCT US97/20014 (which are all herein incorporated by reference). Briefly, previous work has shown that electron transfer can proceed rapidly through the stacked x-orbitals of double stranded nucleic acid, and significantly more slowly through single-stranded nucleic acid. Accordingly, this can serve as the basis of an assay. Thus, by adding ETMs to a nucleic acid that is attached to a detection electrode via a conductive oligomer, electron transfer between the ETM and the electrode, through the nucleic acid and conductive oligomer, may be detected.

### Detection Electrodes

In an embodiment, the detection electrodes are formed on a substrate, typically formed of gold electrodes. However, as will be appreciated by those in the art, other electrodes can be used as well. The substrate can comprise a wide variety of materials, as will be appreciated by those in the art. In one embodiment, the substrate comprises a printed circuit board (PCB). Thus, in general, the suitable substrates include, but are not limited to, fiberglass, teflon, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), etc.

In general, materials include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g., all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

Accordingly, in some embodiments, disclosed are biochips (sometimes referred to herein “chips”) that comprise substrates comprising a plurality of electrodes, such as gold electrodes. The number of electrodes forms arrays. Each electrode can comprise a self-assembled monolayer as outlined herein and known to a skilled artisan. In an embodiment, one of the monolayer-forming species comprises a capture ligand as outlined herein and known to a skilled artisan. In addition, each electrode has an interconnection that is attached to the electrode at one end and is ultimately attached to a device that can control the electrode. That s, each electrode is independently addressable.

The substrates can be part of a larger device comprising a detection chamber that exposes a given volume of sample to the detection electrode. Generally, the detection chamber ranges from about 1 μl to 1 ml, or about 10 μl to 500 μl. As will be appreciated by those in the art, depending on the experimental conditions and assay, smaller or larger volumes may be used.

In some embodiments, the detection chamber and electrode are part of a cartridge that can be placed into a device comprising electronic components (an AC/DC voltage source, an ammeter, a processor, a read-out display, temperature controller, light source, etc.). In this embodiment, the interconnections from each electrode are positioned such that upon insertion of the cartridge into the device, connections between the electrodes and the electronic components are established.

Detection electrodes on circuit board material (or other substrates) are generally prepared in a wide variety of ways. In general, high purity gold is used, and it may be deposited on a surface via vacuum deposition processes (sputtering and evaporation) or solution deposition (electroplating or electroless processes). When electroplating is done, the substrate must initially comprise a conductive material; fiberglass circuit boards are frequently provided with copper foil. Frequently, depending on the substrate, an adhesion layer between the substrate and the gold in order to insure good mechanical stability is used. Thus, some embodiments utilize a deposition layer of an adhesion metal such as chromium, titanium, titanium/tungsten, tantalum, nickel or palladium, which can be deposited as above for the gold. When electroplated metal (either the adhesion metal or the electrode metal) is used, grain refining additives, frequently referred to in the trade as brighteners, can optionally be added to alter surface deposition properties. Brighteners are mixtures of organic and inorganic species such as cobalt and nickel.

In general, the adhesion layer is from about 100 Å thick to about 25 microns (1000 microinches). If the adhesion metal is electrochemically active, the electrode metal must be coated at a thickness that prevents “bleed-through”; if the adhesion metal is not electrochemically active, the electrode metal may be thinner. Generally, the electrode metal (gold) is deposited at thicknesses ranging from about 500 Å to about 5 microns (200 microinches), with from about 30 microinches to about 50 microinches. In general, the gold is deposited to make electrodes ranging in size from about 5 microns to about 5 mm in diameter or from about 100 to 250 microns. The detection electrodes thus formed are then cleaned and SAMs added, as is discussed below.

Thus, provided are methods of making a substrate comprising a plurality of gold electrodes. The methods first comprise coating an adhesion metal, such as nickel or palladium (optionally with brightener), onto the substrate. The electrode metal, such as gold, is then coated via electroplating onto the adhesion metal. Then the patterns of the device, comprising the electrodes and their associated interconnections are made using lithographic techniques, particularly photolithographic techniques as are known in the art, and wet chemical etching. Frequently, a non-conductive chemically resistive insulating material such as solder mask or plastic is laid down using these photolithographic techniques, leaving only the electrodes and a connection point to the leads exposed; the leads themselves are generally coated.

### Self-Assembled Monolayers

The methods may continue with the addition of SAMs (not pictured in FIGS. 1a and 1b), which is also attached to the electrode surface in some embodiments via one or more linkers in similar format to the capture probe linkers (element 2a in FIGS. 1a and 1b), and which serves to prevent or lessen undesired electron transfer events to the electrode surface. In some embodiments, drop deposition techniques are used to add the required chemistry, i.e., the monolayer forming species, one of which is a capture ligand. Drop deposition techniques are well known for making “spot” arrays. This is done to add a different composition to each electrode, i.e., to make an array comprising different capture ligands. Alternatively, the SAM species may be identical for each electrode, and this may be accomplished using a drop deposition technique or the immersion of the entire substrate or a surface of the substrate into the solution.

The present system finds particular utility in array formats, i.e., wherein there is a matrix of addressable detection electrodes (herein generally referred to “pads,” “addresses” or “micro-locations”). By “array” herein is meant a plurality of capture ligands in an array format; the size of the array will depend on the composition and end use of the array. Arrays containing from about 2 different capture ligands to many thousands can be made. Generally, the array will comprise from two to as many as 100,000 or more, depending on the size of the electrodes, as well as the end use of the array. Ranges are from about 2 to about 10,000, about 5 to about 1000, and from about 10 to about 100. In some embodiments, the compositions may not be in array format; that is, for some embodiments, compositions comprising a single capture ligand may be made as well. In addition, in some arrays, multiple substrates may be used, either of different or identical compositions. Thus, for example, large arrays may comprise a plurality of smaller substrates.

In an embodiment, although in many systems this is not required, the electrode comprises a self-assembled monolayer (SAM). As outlined herein, the efficiency of target anatyte binding (for example, oligonucleotide hybridization) may increase when the analyte is at a distance from the electrode. Similarly, non-specific binding of biomolecules, including the target analytes, to an electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent Such contact can result in a direct “short circuit” or an indirect short circuit via charged species which may be present in the sample. Accordingly, the monolayer is tightly packed in a uniform layer on the electrode surface, such that a minimum of “holes” exist. The monolayer thus serves as a physical barrier to block solvent accessibility to the electrode.

The SAM may comprise conductive oligomers alone, or a mixture of conductive oligomers and insulators. As outlined herein, the use of a monolayer reduces the amount of non-specific binding of biomolecules to the surface, and, in the case of nucleic acids, increases the efficiency of oligonucleotide hybridization as a result of the distance of the oligonucleotide from the electrode. Thus, a monolayer facilitates the maintenance of the target analyte away from the electrode surface. In addition, a monolayer serves to keep charge carriers away from the surface of the electrode. Thus, this layer helps to prevent electrical contact between the electrodes and the electronic transfer moieties (ETMs; redox-active), or between the electrode and charged species within the solvent. Such contact can result in a direct “short circuit” or an indirect short circuit via charged species which may be present in the sample. Accordingly, the monolayer is tightly packed in a uniform layer on the electrode surface, such that a minimum of “holes” exist. The monolayer can thus serve as a physical barrier to block solvent and undesired signal (“noise”) accessibility to the electrode.

### Nucleic Acid Testing with a Single-Use Device

An exemplary process is generally described. Although the various elements (steps) are discussed as sequential steps having a prescribed order, it should be understood that the process is exemplary and not intended to be limiting. Persons of ordinary skill will recognize that many of the various elements (steps) can be performed in different orders than described herein, can be performed simultaneously or substantially simultaneously with other elements (steps), or can be omitted altogether. Thus, the order of the elements (steps) discussed is not limiting.
Step 1: load sample.
Step 2: extract nucleic acids, such as, e.g., DNA.
Step 3: combine nucleic acids, such as, e.g., DNA with amplification reagents including ETM-labeled primer (i.e., signal primer).
Step 4: amplify nucleic acids, such as, e.g., DNA to produce a double stranded signal amplicon.
Step 5: incubate the double stranded signal amplicon with exonuclease to form single stranded signal amplicon.
Step 6: combine single stranded signal amplicon with a capture probe.
Step 7: electro-sensor detection.

In some embodiments, the process in Step 2 may include extracting RNA and may further include Step 3a reverse transcribing the RNA to provide cDNA.

### PCR Generally

The polymerase chain reaction (PCR) is a relatively simple technique that amplifies a DNA template to produce specific DNA fragments in vitro. A typical amplification reaction includes target DNA, a thermostable polymerase (such as, e.g., a DNA polymerase), two oligonucleotide primers (5′ and 3′), deoxynucleotide triphosphates (dNTPs), reaction buffer and a divalent cation (such as, e.g., Mg²⁺ and/or Mn²⁺). Each cycle of PCR includes steps for template denaturation, primer annealing and primer extension. The initial step denatures the target DNA by heating it. In the denaturation process, the two intertwined strands of DNA separate from one another, producing the necessary single-stranded DNA template for replication by the thermostable DNA polymerase. In the next step of a cycle, the temperature is reduced so that the oligonucleotide primers can form stable associations (anneal) with the denatured target DNA and serve as primers for the DNA polymerase. Finally, the synthesis of new DNA begins. An enzyme called “Taq polymerase” synthesizes (“builds”) two new strands of DNA, using the original strands as templates. This process results in the duplication of the original DNA, with each of the new molecules containing one old and one new strand of DNA. Then each of these strands can be used to create two new copies, and so on. The cycle of denaturing and synthesizing new DNA is repeated as many as 30 or 40 times, leading to more than one billion exact copies of the original DNA segment. The cycling process of PCR is typically automated in a thermocycler, which is programmed to alter the temperature of the reaction to allow DNA denaturing and synthesis.

There are several nucleic acid amplification technologies that differ from traditional PCR reactions in that the reaction is run at a single temperature. These isothermal methods include the cycling probe reaction, strand displacement, Invader™, SNPase, rolling circle reaction and NASBA. The methods and systems described herein can also be used with isothermal methods.

In some embodiments, in the absence of nucleic acid synthesis, the primer cannot bind to the capture probe and there should be little or no electron transfer between the signal primer and the electrode. In other embodiments, in the absence of nucleic acid synthesis, the primer can bind to the capture probe but the electron transfer is either undetectable or below a predefined limit.

The selection of suitable PCR conditions is within the purview of one of ordinary skill in the art. Those skilled in the art will appreciate that it may be necessary to adjust the concentrations of the nucleic acid target, primers and temperatures of the various steps in order to optimize the PCR reaction for a given target and primer. Such optimization does not entail undue experimentation.

### Types of PCR

The signal primers disclosed herein may be used in any amplification reaction including PCR, 5-RACE, Anchor PCR, “one-sided PCR,” LCR, NASBA, SDA, RT-PCR, real-time PCR, quantitative PCR, quantitative RT-PCR, and other amplification systems known in the art including in a universal primer format.

In some embodiments, the signal primer has two regions: one capable of binding to the DNA template during PCR amplification as described in FIG. 2a item ‘i’ and a second capable of producing a signal during redox mediated electronic detection as described in FIG. 2a item ‘ii’. In some embodiments, the signal primer comprises three regions: one capable of binding to the DNA template during PCR amplification (FIG. 21 item ‘i’), a second capable of producing a signal during redox mediated electronic detection (FIG. 21 item ‘ii’), and a third linking the first and second regions (FIG. 21, linker symbolized as a triangle).

The sequence the signal primers amplify is selected based upon a known target sequence, such that if hybridization to a complementary target sequence occurs amplification proceeds at an appreciable and detectable rate.

PCR reactions can involve drastic temperature swings. Herein, it was not known if the signal primer could withstand these temperature swings. For example, high temperatures (e.g., 90 to 99° C.) used to denature double stranded DNA, lower temperatures (e.g., 40 to 60° C.) at which DNA primers hybridize to the DNA template, and hybridization temperatures (e.g., 60 to 80° C.).

### Assay Development

When developing assays, which utilize a prior art sandwich assay (capture probe bound to an amplicon bound to a signal probe), it can be difficult to find portions of the target organism sequence that are highly conserved (i.e., detectable across many variants) but also uniquely identify the target organism of interest. This is even more complicated when the highly conserved but unique identifier must be long enough to allow both capture and signal probe binding. A key advantage of the methods and systems provided is that there is no need to develop a signal probe. Stated another way, there is no need to develop an amplicon with a signal probe binding region. As such, the amplicons produced can be much shorter making the design of assays simpler and faster. This should result in shorter extension times, sharper melting points, and overall higher efficiency in each round of amplification since the amount of synthesis is reduced compared to traditional sandwich assay systems. This could also lead to higher detection signal and thus higher sensitivity.

Described is an assay, which does not utilize a signal probe. Described is an assay which does not utilize a sandwich assay during detection.

Because unbound signal primer is driven away from the electrode when voltage is applied there is no need to separate the signal primer from the single stranded signal amplicon. Unlike fluorescence-based systems, which require the use of a quencher, the present system does not require the use of such donor/quencher dye pairs.

### Design of Synthetic Oligonucleotides

Regarding the design of synthetic oligonucleotides for use in amplification reactions, Rychlik et al., (1989, Nucleic Acids Research, vol 17(21):8543-8551) and Rychlik (1995, Molecular Biotechnology, vol 3:129-134), describe selection criteria and computer programs to design probes and primers, including primers for in vitro amplification of DNA. Both teach that primers should not generate secondary structure or exhibit self-hybridization. U.S. Pat. No. 6,495,323 (incorporated by reference in its entirety) describes in detail the formation of probes attached to an electron transfer moiety. The same process to make a ferrocene labeled probe can be used to make a ferrocene labeled primer.

### Probe Synthesis, Functionalization and Conjugation-Generally

U.S. Pat. No. 10,001,476 discloses in more detail probe synthesis, functionalization and conjugation and its disclosure is incorporated by reference in its entirety. Probe synthesis, functionalization and conjugation are all well-known techniques in the art.

Nucleic acid capture probes are typically designed to be complementary to a roughly 40- to 50-base sequence within the target nucleic acid. The capture probe sequence is usually complementary to the 3′-region of the target nucleic acid (but the reverse, i.e., the 5′-region of the target nucleic acid can also be true), and is designed to have a melting temperature (TM) of about 50° C. Capture probes can be modified either at the 3′ end or the 5′ end with a disulfide linker for covalent attachment to a gold electrode surface, e.g., as essentially described in commonly owned U.S. Pat. No. 6,753,143 and U.S. Pat. No. 7,820,391, each of which is herein incorporated by reference in their entireties.

Capture probes, including, e.g., nucleic acids, can be adhered to electrodes or other substrate surfaces directly or indirectly, covalently or non-covalently, using a variety of well-known techniques. See, e.g., Ch. 13, Chemically Modified Electrodes, Martin and Foss, pp. 403-442, Laboratory Techniques in Electroanalytical Chemistry; 2d Ed., Kissinger and Heineman, Eds., MARCEL DEKKER, INC. (1996); Biochip Technology, Cheng and Kricka, Eds. George H. Buchanan Printing Company, Bridgeport, N.J. (2001). In some embodiments, this is done by mixing disulfide self-assembling monolayer insulator sequence precursors along with a disulfide group-bearing 3′ or 5′ modified nucleic acid capture probe as described above and spotting onto gold or gold-plated electrodes. This is mediated by a linker/functional group, e.g., W330, as referenced and described in U.S. Pat. No. 7,820,391 (incorporated by reference in its entirety), or N150 as referenced and described in U.S. Pat. No. 6,753,143 (incorporated by reference in its entirety). As the person of skill will appreciate, there are many types of linkers available, e.g., as described in the preceding referenced documents.

### Signal Primer Synthesis

The signal primer sequence(s) is/are complementary to (a) specific region(s) of the target. If sequence discrimination is needed, the sequence polymorphism should be as close as possible to the center of the amplicon sequences, and the T_{M}'s of the two amplicons should be as closely matched as possible.

When making the signal primer, the electron transfer moiety (ETM) can be covalently attached to the nucleic acid in a variety of positions: the 5′-end (FIG. 2c), the middle of the sequence (FIG. 2b), or at both (FIG. 2a). In an embodiment, the attachment is via attachment to the base of the nucleoside, or via attachment to the backbone of the nucleic acid, including either to a ribose of the ribose-phosphate backbone or to a phosphate moiety. In embodiments, the compositions are designed such that the electron transfer moieties are as close to the “x-way” as possible.

In some embodiments, the signal primers are designed to have a ferrocene label (such as, e.g., N6, QW56 or QW80). Attachment of the ferrocene label should not perturb the Watson-Crick base pairing of the primer to which the electron transfer moiety is attached, will not disrupt the annealing of primer to the target. In other embodiments, the primer may contain an extra terminal nucleoside at an end of the nucleic acid (n+1 or n+2), which are used to covalently attach the electron transfer moieties (ETMs) but which do not participate in base pair hybridization similar to what is shown in FIG. 2j and FIG. 2k. In yet other embodiments, it may be desirable to insert a linker arm that separates the electron transfer moiety from the primer region as shown in FIGS. 2l. and 2m. In yet other embodiments, it may be desirable to have more than one signal moiety on the primer region as shown in FIG. 2n. In some embodiments, one ferrocene label is added to the signal primer sequence. In some embodiments, two ferrocene labels are added to the signal primer sequence. In some embodiments, three ferrocene labels are added to the signal primer sequence. In some embodiments, four ferrocene labels are added to the signal primer sequence. In some embodiments, five ferrocene labels are added to the signal primer sequence. In some embodiments, six ferrocene labels are added to he signal primer sequence. In some embodiments, seven ferrocene labels are added to the signal primer sequence. In some embodiments, eight ferrocene labels are added to the signal primer sequence. In some embodiments, nine ferrocene labels are added to the signal primer sequence. In some embodiments, ten ferrocene labels are added to the signal primer sequence. In some embodiments, one to ten ferrocene labels are added to the signal primer sequence. In some embodiments, one to six ferrocene labels are added to the signal primer sequence. In some embodiments, six ferrocene labels are added per signal primer.

In some embodiments, the signal primers are designed to have a ferrocene label (such as, e.g., N6, QW56 or QW80) at the 5′-end or close to the 5′-end (e.g., attached to any one nucleotide of nucleotides 1 to 3 at the 5′-end of the signal or attached to any one nucleotide of nucleotides 1 to 2 at the 5′-end) of the non-phosphorylated oligonucleotide signal primers. In some embodiments, ferrocene label(s) are added to the 5′-terminus of the signal primer sequence(s). In some embodiments, one ferrocene label is added to the 5′-terminus of the signal primer sequence. In some embodiments, two ferrocene labels are added to the 5′-terminus of the signal primer sequence. In some embodiments, three ferrocene labels are added to the 5′-terminus of the signal primer sequence. In some embodiments, four ferrocene labels are added to the 5′-terminus of the signal primer sequence. In some embodiments, five ferrocene labels are added to the 5′-terminus of the signal primer sequence. In some embodiments, six ferrocene labels are added to the 5′-terminus of the signal primer sequence. In some embodiments, seven ferrocene labels are added to the 5′-terminus of the signal primer sequence. In some embodiments, eight ferrocene labels are added to the 5′-terminus of the signal primer sequence. In some embodiments, nine ferrocene labels are added to the 5′-terminus of the signal primer sequence. In some embodiments, ten ferrocene labels are added to the 5′-terminus of the signal primer sequence. In some embodiments, one to ten ferrocene labels are added to the 5′-terminus of the signal primer sequence. In some embodiments, one to six ferrocene labels are added to the 5′-terminus of the signal primer sequence. In some embodiments, six ferrocene labels are added per signal primer. Since all hybridization reactions must take place at a single temperature, the Tu values of all signal primers should be within a range of 5° C. Since all detection reactions must occur within the same solution, signal primers and capture probes must be designed to avoid any cross-hybridization; maximum ΔG₀ values for cross-hybridization have been empirically established.

The signal primer are synthesized using standard phosphoramidite chemistry and can include any nucleotide or modified base, which is amenable to DNA polymerase. The nucleic acid portion of the signal primer can be DNA or RNA or chimeric mixtures or derivatives or modified versions thereof. In addition to being labeled with an electrochemically detectable label, the signal primer can be modified at the base moiety, sugar moiety, or phosphate backbone, and may include other appending groups or labels.

The signal primer can be any suitable size but ideally does not bind to the capture probe without extension. In some embodiments, the signal primer(s) are in the range of 10-100 nucleotides or 10-80 nucleotides, or 11-40 nucleotides, or 17-25 nucleotides although signal primer(s) may be longer or shorter depending upon the need.

The signaling portion of the signal primers may comprise at least one or more electron transfer moieties selected from the group including, but not limited to, ferrocene and ferrocene derivatives. In one embodiment, the signaling portion of the signal primers may comprise one to six electron transfer moieties. In one embodiment, the signaling portion of the signal primers may comprise one to ten electron transfer moieties.

In some embodiments, the signal primer or single stranded signal amplicon or double stranded signal amplicon may contain one or multiple labels (which may be the same or different). In one aspect, the oligonucleotides are labeled and the label is any moiety, which undergoes a detectable electrochemical change upon hybridization with a capture probe on an electrode surface. In an embodiment, the label is a ferrocene tag and the label undergoes a detectable change in one or more electrochemical properties. Such properties include, but are not limited to, signal intensity, electrochemical potentials, or reaction constants.

The signaling portion of the signal primers may be located at one or more positions within the signal primer and/or at or near the 5′-termini. In one embodiment, the signaling portion of the signal primers may be located on the first or second 3′- or 5′-most terminal nucleotides, the first or second or third 3′- or 5′-most terminal nucleotides, one of the first five 3′- or 5′-most terminal nucleotides, one of the first ten 3′- or 5′-most terminal nucleotides, one of the first fifteen 3′- or 5′-most terminal nucleotides, or one of the first twenty 3′- or 5′-most terminal nucleotides. In a specific embodiment, the signaling portion of the signal primers is located on the first base of the 3′- or 5′-terminus.

The signal primers are devoid of enhancing groups. They do not undergo a detectable change in any observable property upon hybridization and/or extension. The signaling portion of the signal primers is not a fluorescent moiety.

The signal primers can be labeled (as described above) using any known labeling method. As an example, the signal primers may be labeled by: (1) attachment at the sulfur of a phosphorothioate linkage; (2) attachment at a 2′-amino group; (3) attachment at the 1′ position using an appropriately modified sugar containing an alkylamine substituted carboxamide, for example; (4) attachment at the 1′ position using an abasic site, for example, and an alkyl diamine as a linker, for example; (5) creation of a structure by reductive alkylation of the adduct formed between an alkyl diamine and an abasic site; (6) incorporation using 4′-thio-2′-deoxyuridine or 4′-thiothymidine; (7) attachment at the 2′-position of 4-thiothymidine or 4-thio-2′-deoxyuridine; (8) attachment at the 4-amino position of deoxycytidine, if the 4-amino group is derivatized with an alkylamine; (9) attachment through the 6′ position of adenine, if the 6-amino group is derivatized with an alkylamino moiety; (10) incorporation using the 8′ position of adenine if this position is substituted with an alkyl thioamine; (11) attachment at the N2 of guanine, if the N2 amino is derivatized with an alkylamino group; or (12) attachment at the N2 position of aminoadenine if the 2-amino group is derivatized with an alkylamine.

All signal primers may be purified using techniques known in the art.

### False Positive Results

False positive results can cause serious problems including the unnecessary use of antibiotics, antimicrobials or antifungal treatments. To avoid false positive results the signal primer should not bind to the capture probe. For example, in FIG. 6 section c is the extension portion of the amplicon and section b is the primer region and section a is the signaling moiety. Section b cannot bind to the capture probe, thus reducing false positives. Additionally, in the method steps, a separation step can be employed to separate signal primers from single stranded signal amplicons.

### False Negative Results

False negative results are rarer. In prior art systems, the amplicon binds to a signal probe. If there is an error in the signal probe function or design, a false negative will result. By eliminating the requirement for the amplicon to bind a signal probe, false negatives can be reduced.

### Signal Primers

Signal primers unlike capture probes used in the system, are not bound to an electrode prior to detection. During detection, they are not directly bound to the electrode but bind a capture probe, which holds them in place for detection. Signal primers unlike signal probes used in prior art systems, are involved in amplification and detection.

In some embodiments, the primer is made of deoxyribonucleic acid, ribonucleic acid, peptide nucleic acid, PEG-modified nucleic acid, hexa-polyethylene glycol modified nucleic acid, chimeric mixtures or derivatives or modified versions thereof.

In some embodiments, the signal primer has no overlap with the capture probe. In some embodiments, the signal primer overlaps with the capture probe by about 1-10 base pairs. In some embodiments, the signal primer overlaps with the capture probe by about 1-12 base pairs.

The signal primers disclosed herein may be better understood by the following embodiments. In one embodiment, a signal primer comprises nucleic acids and a detectable label. In some embodiments, a signal primer comprises nucleic acids and an electron transfer moiety. In certain embodiments, a signal primer comprises nucleic acids and a ferrocene label. In another embodiment, a signal primer comprises nucleic acids and an electron transfer moiety, wherein the electron transfer moiety is attached to the 5′-end of the primer. In some embodiments, a signal primer comprises nucleic acids and a label capable of producing a signal during redox mediated electronic detection. In some embodiments, a signal primer comprises nucleic acids and an electrochemically detectable label. In some embodiments, a signal primer comprises 5-12 nucleic acids and an electrochemically detectable label. In some embodiments, a signal primer comprises 5-25 nucleic acids and an electrochemically detectable label. In some embodiments, a signal primer is a self-annealing primer and comprises a electrochemically detectable label. In some embodiments, a signal primer comprises nucleic acids and an electron transfer moiety, wherein the electron transfer moiety is attached to the 5′-end of the primer and is capable of producing a signal during redox mediated electronic detection. In some embodiments, a signal primer comprises nucleic acids and an electrochemically detectable label, wherein the electrochemically detectable label is attached to the 5′-end of the primer. In some embodiments, a signal primer consists of nucleic acids and an electrochemically detectable label.

### Determining Target Concentration

When electrochemical detection is used, the main objective of the nucleic acid amplification step is to generate about a 0.01 picomolar concentration of detectable nucleic acid from the target molecule, as it has been found that this is in the range of the lower detection limit for electrochemical detection. If, as is known, one microliter of blood contains about 5×10³ molecules of DNA, then one milliliter, which is a reasonably accessible sample volume, contains 5×10⁶ molecules, or roughly about 10⁷ molecules. To go from the amount of DNA in 1 ml of blood to 0.01 pmol of DNA requires an amplification of about 10³ fold. This is certainly achievable using several well-known amplification techniques. Performing a similar calculation, for a different sample types and sample volumes, to determine the degree of amplification will be apparent to those skilled in the art.

### Removal of Primers After Amplification

It has been found that a consequence of seeking to develop systems incorporating rapid PCR reactions, i.e., completed amplification in less than about 10 minutes, that it is necessary to increase the primer concentrations. As discussed above, if excess primers are in the mix during detection, this can generate a “primer background” in the detection step, which can interfere with or reduce signal generation of the single stranded signal amplicon bound to the capture oligonucleotide. Co-owned U.S. Pat. No. 10,864,522 which is herein incorporated by reference in its entirety describes a processing cartridge and a method for detecting a pathogen in a sample. Specifically, the U.S. Pat. No. 10,864,522 patent describes moving sample, primers and amplicons on a digital microfluidic path. In such systems, the primer and amplicon will have different charge-to-mass ratios such that moving them along the digital microfluidic path can separate them from one another. The PCR double stranded amplified material is moved through the digital microfluidic path, with the smaller synthetic oligonucleotide primers moving the fastest and the larger amplicons moving slower. After a certain amount of time, the PCR amplified material can be split on the digital microfluidic path with oligonucleotide primers in one half and the larger amplicons in the other. In some embodiments, the splitting occurs after the PCR amplified material has been denatured with endonuclease. In one embodiment, a membrane or gel can be added to the digital microfluidic path that slows large molecules compared to short to aid in splitting the material.

### Detectable Label

The detectable label can include electron transfer moieties. Electron transfer moieties include ferrocene labels. In some embodiments, the ferrocene label (such as, e.g., N6, QW56 or QW80) is on the 5′-end of the primer or close to the 5′-end of the primer (e.g., attached to any one nucleotide of nucleotides 1 to 3 at the 5′-end or attached to any one nucleotide of nucleotides 1 to 2 at the 5′-end). However, the ferrocene label can also be on the 3′-end of the primer or both the 5′-and 3′-end. The signal primers produce a double stranded signal amplicon. When the double stranded signal amplicon is denatured, it forms a single stranded signal amplicon, which can bind a capture probe and generate an electrochemical signal. As such, signal primers play a role both, in amplification and detection. Signal primers allow the system to utilize shorter signal amplicons. In some embodiments, the amplicon is about 60 base pairs. In some embodiments, the amplicon is about 30-120 base pairs. In some embodiments, the amplicon is about 70-250 base pairs. In some embodiments, the amplicon is about 50-500 base pairs.

In one embodiment, a nucleic acid is modified with at least one electron transfer moiety at one location. In one embodiment, a nucleic acid is modified with at least two electron transfer moieties at two locations (see FIG. 2n). In one embodiment, a nucleic acid is modified with more than two electron transfer moieties at more than two locations. In one embodiment, a nucleic acid is modified with a plurality of electron transfer moieties at a plurality of locations. For example, to increase the signal obtained from the primer, a plurality of electron transfer moieties at a plurality of locations may be used. For example, the electron transfer moieties can be attached both 5′ and 3′, as is generally depicted in FIG. 2n. In one embodiment, the plurality of electron transfer moieties are the same, to result in a uniform signal. In another embodiment, each of the plurality of electron transfer moieties may be different. In one embodiment, the two electron transfer moieties are the same, to result in a uniform signal. In another embodiment, the two electron transfer moieties may be different.

In some embodiments, the detectable label is held one base pair away from the capture probe and still produces a detectable signal. In some embodiments, the detectable label is held anywhere from 1-10 base pairs away from the capture probe and still produces a detectable signal. In some embodiments the detectable label is held anywhere from 1-50 base pairs away from the capture probe and still produces a detectable signal. In some embodiments the detectable label is held anywhere from 1-100 base pairs away from the capture probe and still produces a detectable signal. In some embodiments the detectable label is held anywhere from 36-72 base pairs away from the capture probe and still produces a detectable signal. Accordingly, a capture probe signal primer complex is provided herein, in which the ETM (such as, e.g., a ferrocene label) is from 1-10 base pairs away from the position to which the capture probe hybridizes to. In some embodiments, a capture probe signal primer complex is provided in which the ETM (such as, e.g., a ferrocene label) is from 1-50 base pairs away from the position to which the capture probe hybridizes to. In further embodiments, a capture probe signal primer complex is provided herein, in which the ETM (such as, e.g., a ferrocene label) is from 1-100 base pairs away from the position to which the capture probe hybridizes to. In further embodiments, a capture probe signal primer complex is provided herein, in which the ETM (such as, e.g., a ferrocene label) is from 36-72 base pairs away from the position to which the capture probe hybridizes to.

### Single Stranded Signal Amplicon

A single stranded signal amplicon is shown in FIG. 2 (d-f), where ‘i’ is the detection moiety, e.g., ferrocene, and ‘i’ is the annealing region, e.g., the region that binds to the capture probe during detection. In some embodiments, the amplicon comprises two regions: one capable of binding to the capture probe and a second capable of producing a signal during redox mediated electronic detection. In some embodiments, the amplicon comprises three regions: one capable of binding to the capture probe, a second capable of producing a signal during redox mediated electronic detection and a third linking the first and second regions.

Allowing PCR to proceed using the disclosed synthetically labeled oligonucleotide primers in the presence of the appropriate target and DNA polymerase with associated components, generates a newly synthesized DNA molecule with an incorporated signal moiety region. This molecule can then be denatured and hybridized to a target sequence on a solid support. The signal moiety region can then be used for generating a signal. In some embodiments, the signal moiety is a ferrocene label.

In some embodiments, the single stranded signal amplicon is 60 bases in length. In some embodiments, the single stranded signal amplicon is 30-60 bases in length. In some embodiments, the single stranded signal amplicon is 20-100 bases in length. In some embodiments, the single stranded signal amplicon is 20-150 bases in length.

In some embodiments, the amplicon is selected from the group deoxyribonucleic acid, ribonucleic acid, peptide nucleic acid, PEG-modified nucleic acid and hexa-polyethylene glycol modified nucleic acid.

In some embodiments, a single stranded signal amplicon comprises nucleic acids and a detectable label. In some embodiments, a single stranded signal amplicon comprises nucleic acids and an electron transfer moiety. In some embodiments, a single stranded signal amplicon comprises nucleic acids and a ferrocene label. In some embodiments, a single stranded signal amplicon comprises nucleic acids and an electron transfer moiety, the nucleic acids comprising a first portion and a second portion, wherein the first portion and second portion are different.

In some embodiments, a single stranded signal amplicon comprises nucleic acids and an electron transfer moiety, the nucleic acids comprising a first portion and a second portion, wherein the first portion and second portion are different and wherein the first portion is capable of binding to a first capture probe and the second portion is capable of binding to a second capture probe. In some embodiments, a single stranded signal amplicon comprises nucleic acids and an electron transfer moiety, the nucleic acids comprising a first portion and a second portion, wherein the first portion and second portion are different and wherein the first portion is capable of binding to a first capture probe but not a second capture probe and the second portion is capable of binding to the second capture probe but not the first capture probe. In some embodiments, a single stranded signal amplicon comprises nucleic acids and an electron transfer moiety, the nucleic acids comprising a first portion and a second portion, wherein the first portion and second portion are different and wherein the first portion is capable of binding to a first capture probe and a second capture probe. In some embodiments, a single stranded signal amplicon comprises nucleic acids and an electron transfer moiety, the nucleic acids comprising a first portion and a second portion, wherein the first portion and second portion are different and wherein the first portion is capable of binding to a first capture probe and a second capture probe and wherein the second portion is capable of binding to a first capture probe and a second capture probe. In certain embodiments, the electron transfer moiety is attached to the 5′-end or close to the 5′-end of the single stranded signal amplicon (e.g., attached to any one nucleotide of nucleotides 1 to 3 at the 5′-end or attached to any one nucleotide of nucleotides 1 to 2 at the 5′-end). In some embodiments, a single stranded signal amplicon comprises an electrochemically detectable label. In some embodiments, a single stranded signal amplicon comprises a capture probe-binding region, a primer-binding region and an electrochemically detectable label region. Herein, in certain embodiments the electrochemically detectable label region is 5-100 base pairs away from the capture probe-binding region. In some embodiments, the electron transfer moiety is an electrochemically detectable label. Herein, in certain embodiments the electron transfer moiety is an electrochemically detectable label and is attached to the 5′-end or close to the 5′-end of the single stranded signal amplicon. In some embodiments, a single stranded signal amplicon consists of a capture probe binding region, a primer binding region and an electrochemically detectable label region. Herein, in certain embodiments the electrochemically detectable label is 5-100 base pairs away from a capture probe binding region. In some embodiments, a single stranded signal amplicon comprises an electrochemically detectable label attached to the 5′-end and 3′-end of the single stranded signal amplicon.

### Double Stranded Amplicon

In some embodiments, the double stranded signal amplicon is 60 base pairs in length. In some embodiments, the double stranded signal amplicon is 30-60 base pairs in length. In some embodiments, the double stranded signal amplicon is 20-100 base pairs in length. In some embodiments, the double stranded signal amplicon is 20-150 base pairs in length.

In some embodiments, a double stranded signal amplicon comprises nucleic acids and a detectable label. In some embodiments, a double stranded signal amplicon comprises nucleic acids and an electron transfer moiety. In some embodiments, a double stranded signal amplicon comprises nucleic acids and a ferrocene label. In some embodiments, a double stranded signal amplicon comprises nucleic acids and an electron transfer moiety, the nucleic acids comprising a first portion and a second portion wherein the first portion and second portion are different. In some embodiments, a double stranded signal amplicon comprises nucleic acids and an electron transfer moiety, wherein the electron transfer moiety is attached to the 5′-end or close to the 5′-end of the double stranded signal amplicon. In some embodiments, a double stranded signal amplicon comprises nucleic acids and an electron transfer moiety, wherein the electron transfer moiety is attached to the forward and reverse end of the double stranded signal amplicon. In some embodiments, a double stranded signal amplicon comprises an electrochemically detectable label.

### Hybridization Complex: Capture Probe and Signal Amplicon

In some embodiments, the forward primer and reverse primer are labeled with an electrochemically detectable label. In some embodiments, the forward primer is labeled with an electrochemically detectable label. In some embodiments, the reverse primer is labeled with an electrochemically detectable label. In some embodiments, there is a first capture probe specific to the forward primer labeled with an electrochemically detectable label and a second capture probe specific to the reverse primer labeled with an electrochemically detectable label.

In some embodiments, a hybridization complex comprises a capture probe bound to an amplicon, the amplicon comprising nucleic acids and a detectable label. In some embodiments, a hybridization complex comprises a capture probe bound to an amplicon, the amplicon comprising nucleic acids and an electron transfer moiety. In some embodiments, a hybridization complex comprises a capture probe bound to an amplicon, the amplicon comprising nucleic acids and a ferrocene label. In some embodiments, a hybridization complex comprises a capture probe bound to an amplicon, the amplicon comprising nucleic acids and an electron transfer moiety, wherein the nucleic acids comprise a first portion and a second portion wherein the first portion and second portion are different. In some embodiments, a hybridization complex comprises a capture probe bound to an amplicon, the amplicon comprising nucleic acids and an electron transfer moiety, wherein the nucleic acids comprise a first portion and a second portion and the capture probe is capable of binding the first portion but not the second portion. In some embodiments, a hybridization complex comprises a capture probe bound to an amplicon, the amplicon comprising nucleic acids and an electron transfer moiety, wherein the nucleic acids comprise a first portion and a second portion and the capture probe is capable of binding the second portion but not the first portion. In some embodiments, a hybridization complex comprises a capture probe bound to an amplicon, the amplicon comprising nucleic acids and an electron transfer moiety, wherein the nucleic acids comprise a first portion and a second portion and the capture probe is capable of binding the second portion and the first portion. In some embodiments, a hybridization complex comprises a capture probe bound to an amplicon, the amplicon comprising nucleic acids and an electron transfer moiety, wherein the hybridization complex is bound to an electrode, the electrode comprising a monolayer.

### Methods

The signal primers disclosed herein can be used in methods of diagnosis, wherein the signal primer is complementary to a sequence (e.g., genomic or cDNA) of an infectious disease agent or is capable of initiating synthesis or amplification of a sequence of an infectious disease agent, e.g., of human disease including, but not limited to, viruses (e.g., HIV, HPV, etc.), bacteria, parasites, and fungi, thereby diagnosing the presence of the infectious agent in a sample from a patient. The type of target nucleic acid can be genomic, cDNA, mRNA, or synthetic, or the source may be human, animal, fungi or bacterial. In another embodiment that can be used in the diagnosis or prognosis of a disease or disorder, the target sequence is a wild type human genomic DNA or RNA, or cDNA sequence, mutation of which is implicated in the presence of a human disease or disorder, or alternatively, can be the mutated sequence. In such an embodiment, the amplification reaction can be repeated for the same sample with different sets of signal primers (for example, with differently labeled signal primers), which selectively identify the wild type sequence or the mutated version. By way of example, the mutation can be an insertion, substitution, and/or deletion of one or more nucleotides, or a translocation. In another embodiment, the signal primers can be used in SNP analysis, pharmacogenomics and toxicogenetics.

In a specific embodiment, disclosed are methods for detecting or measuring a product of a nucleic acid amplification or synthesis reaction comprising: (a) contacting a sample comprising or suspected of comprising one or more target nucleic acid molecules with one or more signal primers (such primers comprise one or multiple labels, which may be the same or different and may be labeled internally, and/or, at or close to the 3′-and/or at or close to the 5′-end), said primers being adapted for use in said amplification or synthesis reaction such that said primers are incorporated into an amplified or synthesized product of said amplification or synthesis reaction when a target sequence or nucleic acid molecule is present in the sample; (b) conducting the amplification or synthesis reaction; and (c) detecting or measuring one or more synthesis or amplification product molecules by redox mediated electronic detection.

In another embodiment, disclosed are methods for detecting a target nucleic acid sequence, comprising contacting a sample containing a mixture of nucleic acids with at least one oligonucleotide, the oligonucleotide being capable of hybridizing a target nucleic acid sequence and comprising at least one detectable moiety, wherein the detectable moiety undergoes a redox reaction after an electrical signal is applied to it, wherein a change in the redox potential indicates the presence of the target nucleic acid sequence. In some embodiments, the target nucleic acid sequence is not separated from the mixture.

In another embodiment, disclosed are methods for quantifying a target nucleic acid molecule, comprising contacting a sample containing a mixture of nucleic acids comprising the target nucleic acid molecule with at least one oligonucleotide containing a detectable moiety, wherein the detectable moiety undergoes a detectable redox reaction upon application of an electrical charge and observing the reaction, wherein the observable reaction is proportional to the amount of the target nucleic acid molecule in the sample.

In another embodiment, disclosed are methods of amplifying one or more nucleic acid molecules comprising (a) mixing one or more templates or target nucleic acid molecules with one or more signal primers; and (b) incubating said mixture under conditions sufficient to synthesize or amplify one or more nucleic acid molecules complementary to all or a portion of said templates or target molecules. In one embodiment, the amplified nucleic acid molecules comprise one or more signal primers. In one aspect, signal primers are incorporated at or close to one or both termini of the synthesized or amplified nucleic acid molecules produced. Disclosed is also one or more nucleic acid molecules produced by such amplification or synthesis reactions.

In another embodiment, disclosed are methods of determining the presence or absence of a target in a sample. In some embodiments, target sequence is a wild type human genomic or RNA or cDNA sequence. In some embodiments, target sequence is a mutated human genomic or RNA or cDNA sequence. The mutation is implicated in the presence of a human disease or disorder. In some embodiments, the signal primer amplifies the wild type target and in others the mutated target. By way of example, the mutation can be an insertion, substitution, and/or deletion of one or more nucleotides, or a translocation. In another embodiment, the signal primers can be used in SNP analysis, pharmacogenomics and toxicogenetics.

In a specific embodiment, disclosed is a method for detecting the presence or absence of a product of a nucleic acid amplification comprising: (a) contacting a sample comprising or suspected to comprise one or more target nucleic acid molecules with one or more signal primers (such primers may comprise one or multiple labels, which may be the same or different and may be labeled internally, and/or, at or near the 3′-and/or at or near the 5′-end), said signal primers being adapted for use in said amplification or synthesis reaction such that said signal primers are incorporated into an amplified product of said amplification reaction when a target sequence or nucleic acid molecule is present in the sample; (b) conducting the amplification reaction; and (c) detecting or measuring one or more amplification product molecules by electrochemical detection.

In another embodiment, disclosed are methods for determining the absence of at least one particular target or template nucleic acid molecule in a sample, comprising: (a) contacting the sample with a signal primer, wherein at least a portion of the signal primer is capable of forming base pairs (e.g., hybridizing) with at least a portion of the target nucleic acid molecule; and (b) incubating the signal primer and the nucleic acid molecule mixture under conditions sufficient to amplify the at least a portion of the target nucleic acid molecule. Under such conditions, the lack of amplification indicates the absence of the particular nucleotide or nucleotides in the sample. In one embodiment, a method of performing a nucleic acid amplification assay is provided comprising (a) combining reagents for a polymerase chain reaction, polymerase (e.g. a DNA polymerase), a target nucleic acid, and a modified primer, said modified primer comprising a detectable label; (b) cycling the mixture of (a) to provide multiple copies of an amplicon incorporating said modified primer; (c) exposing the mixture in (b) to an exonuclease to produce a single stranded amplicon; (d) exposing said mixture in (c) to a capture oligonucleotide complementary to said single stranded signal amplicon incorporating said modified primer; (e) hybridizing said single stranded signal amplicon incorporating said modified primer, with a capture oligonucleotide; and (f) detecting said label associated with said hybridization. In some embodiments, said target nucleic acid is selected from the group consisting of deoxyribonucleic acid and ribonucleic acid and modifications and derivatives thereof. In some embodiments, said target nucleic acid is extracted from blood, a buccal swab, tissue, a bodily fluid, an environmental sample, a surface of a material, a plant, an animal, a bacteria or a fungi. In some embodiments, said polymerase is a DNA polymerase and is Taq polymerase or thermococcus kodakiensis polymerase. In some embodiments, said primer is selected from the group deoxyribonucleic acid, ribonucleic acid, peptide nucleic acid, PEG-modified nucleic acid and hexa-polyethylene glycol modified nucleic acid. In some embodiments, said detectable label is an electron transfer moiety. In some embodiments, said detectable label is a ferrocene label. In certain embodiments, said detectable label is N6, QW56 or QW80. In some embodiments, cycling is isothermal. In some embodiments, cycling is between a first and second temperature. In some embodiments, said capture oligonucleotide is immobilized on a gold surface. In some embodiments, said capture oligonucleotide is immobilized on an electrode surface. In some embodiments, said detection is electrochemical. In some embodiments, said label is not exposed to a second moiety, which has a second label or a quencher.

In another embodiment, a method of performing a nucleic acid amplification assay is provided, comprising (a) combining PCR reagents, polymerase (e.g. a DNA polymerase), a target nucleic acid or a sample suspected to comprise a target nucleic acid, a modified primer and a second unmodified primer, said modified primer comprising a ferrocene label; (b) cycling the mixture of (a) to provide multiple copies of a first amplicon incorporating said modified primer and a second amplicon incorporating said second unmodified primer if target nucleic acid is present in the sample; (c) exposing the mixture in (b) to an exonuclease to produce a first single stranded signal amplicon incorporating said modified primer and a second single stranded amplicon incorporating said unmodified primer; (d) exposing the mixture in (c) to a capture oligonucleotide complementary to said single stranded signal amplicon incorporating said modified primer; (e) hybridizing said single stranded signal amplicon of said amplicon incorporating said modified primer, with said capture oligonucleotide; and (f) detecting said label associated with said modified primer incorporated into said amplicon. In some embodiments, said target nucleic acid is selected from the group consisting of deoxyribonucleic acid and ribonucleic acid and modifications and derivatives thereof. In some embodiments, said target nucleic acid is extracted from blood, a buccal swab, tissue, a bodily fluid, an environmental sample, a surface of a material, a plant, an animal, a bacteria or a fungi. In some embodiments, said reagents comprise reagents for a polymerase chain reaction amplification. In some embodiments, said polymerase is a DNA polymerase and is Taq polymerase. In some embodiments, said polymerase is a DNA polymerase and is thermococcus kodakiensis polymerase. In some embodiments, said primer is selected from the group deoxyribonucleic acid, ribonucleic acid, peptide nucleic acid, PEG-modified nucleic acid and hexa-polyethylene glycol modified nucleic acid. In some embodiments, said detectable label is selected from the group consisting of N6, QW56 and QW80. In some embodiments, said detectable label is ferrocene, methylene blue or osmium. In some embodiments, cycling is isothermal. In some embodiments, cycling is between a first and second temperature. In some embodiments, said capture oligonucleotide is immobilized on a gold surface. In some embodiments, said capture oligonucleotide is immobilized on an electrode. In some embodiments, the single stranded signal amplicon incorporating the modified primer hybridizes to the capture oligonucleotide. In some embodiments, said detection is electrochemical.

### Integrated Nucleic Acid Test Cartridge

Integrated multiplex target analysis systems are known and are described in U.S. Pat. No. 10,864,522 which is incorporated by reference.

Provided herein are integrated nucleic acid test cartridges capable of performing amplification and detection. Generally, the integrated nucleic acid test cartridges is capable of receiving a sample, extracting DNA, combining DNA with amplification reagents including ETM-labeled primer, amplifying DNA, incubating signal amplicon with exonuclease to form single stranded signal amplicon, combining single stranded signal amplicon with capture probe and electro sensor detection.

In certain embodiments, the target nucleic acid is selected from the group consisting of deoxyribonucleic acid and ribonucleic acid and modifications and derivatives thereof and in others, the target nucleic acid is extracted from blood, a buccal swab, tissue, a bodily fluid, an environmental sample, a surface of a material, a plant, an animal, a bacteria and a fungi.

Further disclosed herein is an integrated nucleic acid test cartridge capable of performing amplification and detection. Further disclosed herein is an integrated nucleic acid test cartridge for use with electrochemical detection. Further disclosed herein is an integrated nucleic acid test cartridge capable of performing amplification. Further disclosed herein is an integrated nucleic acid test cartridge capable of performing detection. Further disclosed herein is an integrated nucleic acid test cartridge capable of performing amplification and detection. Further disclosed herein is an integrated cartridge for nucleic acid testing that operates in conjunction with a reader instrument.

### Kits

Disclosed are kits for the detection of nucleic acid molecules in a sample. Such kits may also be designed to detect nucleic acid molecules of interest during or after nucleic acid amplification reactions. Such kits may be diagnostic kits wherein the presence of the nucleic acid is correlated with the presence or absence of a disease or disorder. Disclosed are kits for carrying out amplification reactions described herein and kits for making the compositions described herein. In specific embodiments, the kits comprise one or more signal primers as defined herein. The kit can further comprise additional components for carrying out the detection assays or other methods. Such kits may comprise one or more additional components selected from the group consisting of one or more polymerases (e.g., DNA polymerases and reverse transcriptases), one or more nucleotides, one or more buffering salts (including nucleic acid amplification buffers), one or more control nucleic acid target molecules (to act as positive controls to test assays), instructions for carry out the methods and the like.

### Examples

### Example 1: Amplification and Detection Using a Signal Primer

SARS-COV-2 amplicons were amplified by either the control primers or the signal primers (FIG. 10). In this example, the reverse primer for SARS-COV2 N1 gene was synthesized with an N6 tag. The concurrent reverse primer, without any tag, was used as the Control. After digestion with A exonuclease, the “Control” amplicons were incubated with SARS-COV-2 N1 signal probes. On the other hand, the amplicons generated by using signal primers were digested with λ exonuclease, but were not incubated with signal probes. The amplicons were then loaded on the detection zone of GenMark's Respiratory Panel cartridges (Carlsbad, CA). The signal generated from amplicons using signal primers was higher than the signal generated using control primers (FIG. 10). Furthermore, no false positive signal was detected from other targets in the detection zone, confirming that the signal primers were not binding to capture probes non-specifically (Fig.11).

Example 2: Detection Using a Traditional Sandwich Assay (Control Primer) vs Signal Primer with the Ferrocene Distanced from the Capture Probe

Adenovirus C, OC43, RSV A and SARS-COV-2 were amplified by either the current reverse primer without any tag (“the control primers”) or the corresponding signal primers. Indicated in FIG. 12 is the base distance between the ferrocene label and the capture probe. In FIG. 6, this is the distance indicated by section b. After digestion with A exonuclease, the “Control” amplicons were incubated with their respective signal probes. Amplicons generated by using signal primers were not digested with λ exonuclease and were not incubated with signal probes. The amplicons were then loaded onto the detection zone of GenMark's Respiratory Panel cartridges (Carlsbad, CA). The signal generated from amplicons using signal primers was generally equal to or higher than the signal generated using control primers (FIG. 12) for the various targets.

1. A method of detecting the presence or absence of a target nucleic acid in a sample, wherein the method comprises the following steps:
(a) combining: (i) a solution comprising target nucleic acid, or suspected to comprise target nucleic acid, with (ii) amplification reagents, to amplify the target nucleic acid if present, wherein the amplification reagents comprises a labeled primer capable of hybridizing to the target nucleic acid;
(b) amplifying the target nucleic acid to produce a double-stranded-labeled amplicon if the target nucleic acid is present;
(c) incubating the double-stranded-labeled amplicon with exonuclease to form a single-stranded-labeled amplicon;
(d) hybridizing the single stranded labeledsingle-stranded-labeled amplicon with a first capture probe; and
(e) detecting the presence or absence or the target nucleic acid in the sample using electrochemical detection.

2. The method of claim 1, wherein the labeled primer, double-stranded-labeled amplicon, and/or single-stranded-labeled amplicon comprises at least one label selected form the group consisting of: ferrocene, methylene blue, and osmium.

3. The method of claim 1, wherein the labeled primer comprises a ferrocene label on the 5′-end, on any one nucleotide of nucleotides 1 to 3 at the 5′-end, on the 3′-end, on any one nucleotide of nucleotides 1 to 3 at the 3′-end or on both, the 5′-and the 3′-end.

4. The method of claim 1, wherein the labeled primer comprises a ferrocene label internal to the labeled primer.

5. The method of claim 1, wherein the labeled primer, double-stranded labeled amplicon, and/or single-stranded-labeled amplicon comprise a plurality of ferrocene labels at least two locations.

6. The method of claim 1, wherein the single-stranded-labeled amplicon comprises a first section capable of hybridizing to the first capture probe, a second section that is not capable of binding to the capture probe, and a third section comprising the at least one label.

7. The method of claim 6, wherein the label is selected from the group consisting of: ferrocene, methylene blue and osmium.

8. The method of claim 1, wherein the labeled primer comprises: (i) a first section capable of hybridizing to the first capture probe, (ii) a second section comprising a linker, and (iii) a third section comprising the at least one label, wherein the linker connects the first section and the third section.

9. The method of claim 1, further comprising separating the labeled primer and the single-stranded-labeled amplicon prior to electrochemical detection.

10. The method of claim 1, further comprising a step of separating the labeled primer and the double-stranded-labeled amplicon prior to electrochemical detection of step (e).

11. The method of claim 1, wherein the labeled primer consists of: (i) a first section capable of hybridizing to the capture probe, and (ii) a second section comprising a first label.

12. A method of detecting the presence or absence of a target nucleic acid in a sample, wherein the method comprises the following steps:
(a) receiving a sample;
(b) extracting nucleic acids from the sample, wherein the nucleic acids are suspected of comprising target nucleic acid;
(c) combining the nucleic acid with amplification reagents to amplify the target nucleic acid, if present, wherein the amplification reagents comprises a signal primer capable of hybridizing to the target nucleic acid;
(d) amplifying the target nucleic acid to produce a double-stranded signal amplicon, if the target nucleic acid is present;
(e) incubating the double-stranded signal amplicon with exonuclease to form a single-stranded signal amplicon;
(f) hybridizing the single-stranded amplicon with a first capture probe; and
(g) detecting the presence or absence of the target nucleic acid in the sample using electrochemical detection.

13. The method of claim 12, wherein the signal primer comprises an electrochemically detectable label.

14. The method of claim 12, wherein the signal primer comprises at least one label selected from the group consisting of: ferrocene, methylene blue, and osmium.

15. The method of claim 12, wherein the signal primer does not bind to the first capture probe.

16. The method of claim 12, wherein a first portion of the single-stranded signal amplicon binds the first capture probe and a second portion of the single-stranded signal amplicon binds a second capture probe.

17. A process for detecting the presence of a single-stranded or double-stranded nucleic acid of interest in a sample, wherein the process comprises the following steps:
(a) providing
(i) a sample suspected of containing the nucleic acid of interest;
(ii) a nucleic acid primer that comprises:
(1) a nucleic acid sequence complementary to at least a portion of the nucleic acid of interest, and
(2) a first electrochemically detectable label;
(iii) reagents for carrying out nucleic acid strand extension;
(b) forming a reaction mixture comprising (i), (ii), and (iii) above;
(c) contacting, under hybridization conditions, the nucleic acid primer with the nucleic acid of interest, if present;
(d) extending the nucleic acid primer, thereby incorporating the electrochemically detectable label into an amplicon to form an electrochemically-labeled amplicon, if said nucleic acid of interest is present;
(e) denaturing the electrochemically-labeled amplicon;
(f) hybridizing the electrochemically-labeled amplicon with a capture probe bound to an electrode surface; and
(g) detecting the presence of the nucleic acid of interest by detecting energy transfer between the electrochemically-labeled amplicon and the electrode surface.

18. The method of claim 17, wherein the electrochemically detectable label is selected from the group consisting of: ferrocene, methylene blue, and osmium.

19. The method of claim 17, wherein the nucleic acid primer comprises: a first portion that binds to the capture probe, and a second portion that does not bind to the capture probe.

20. The method of claim 17, wherein the nucleic acid primer further comprises a second electrochemically detectable label, and wherein the first electrochemically detectable label and the second electrochemically detectable label are different.
